(19) United States
(12) Patent Application Publication
(10) Pub. No.: US 2026/0220784 A1
(43) Pub. Date: Jul. 30, 2026

(54) SYSTEMS AND METHODS FOR ANALYZING MR IMAGES DEPICTING RESIDUAL BREAST TISSUE

(71) Applicant: Sheba Impact Ltd., Ramat Gan (IL)

(72) Inventors: Orit KAIDAR-PERSON, Haifa (IL); Debbie ANABY, Herzliya (IL); Miriam SKLAIR-LEVY, Mevasseret Zion (IL); David SHAVIN, Zur Hadassah (IL); Kfir COHEN, Jerusalem (IL)

(73) Assignee: Sheba Impact Ltd., Ramat Gan (IL)

(21) Appl. No.: 19/141,401

(22) PCT Filed: Dec. 4, 2023
(86) PCT No.: PCT/IL2023/051240

§ 371 Date: Jun. 20, 2025

(30) Foreign Application Priority Data
Dec. 22, 2022 (IL) 299436

(51) Int. Cl. G06T 7/12; A61B 5/00; G06T 7/00; G06T 7/136; G06T 7/62; G16H 50/30

(52) U.S. Cl. CPC G06T 7/12; A61B 5/004; G06T 7/0016; G06T 7/136; G06T 7/62; G16H 50/30; G06T 2207/10088; G06T 2207/20021; G06T 2207/20081; G06T 2207/30052; G06T 2207/30068

(57) ABSTRACT

There is provided a method of automatically segmenting a magnetic resonance (MR) image, comprising: segmenting a residual breast from an input MR image depicting the residual breast with implant after surgical removal of breast tissue, wherein the segmentation of the residual breast includes breast tissue remaining after the surgical removal, feeding the segmented residual breast into a fibroglandular tissue (FGT) segmentation ML model, and obtaining a segmentation of FGT from the segmented residual breast as an outcome of the FGT segmentation ML model. Optionally, the FGT segmentation ML model is trained on a FGT segmentation training dataset of FGT segmentation records, each FGT segmentation record including a MR image of a segmented whole breast of a control subject that excludes an implant and on which no surgical removal of breast tissue has been performed, and a ground truth comprising a segmentation of the FGT depicted in the segmented whole breast.

### RELATED APPLICATION

This application claims the benefit of priority of Israel Patent Application No. 299436 filed on Dec. 22, 2022, the contents of which are incorporated herein by reference in their entirety.

### BACKGROUND

The present invention, in some embodiments thereof, relates to magnetic resonance (MR) images and, more specifically, but not exclusively, to systems and methods for analyzing MR images depicting residual breast tissue.

Breast cancer is the most common cancer in women worldwide. Active screening for breast cancer is recommended (from age of 40-50 years or pending on risk). The goal of screening is to find breast cancer at an early stage when it can be treated and may be cured. Mammography is the most common test used for screening, and MR imaging may be used to screen women who are at high risk for breast cancer. Due to screening programs, most patients are diagnosed in a non-metastatic stage. Surgical resection (breast conserving surgery or mastectomy) is part of the treatment for non-metastatic breast cancer patients. Surgery for breast cancer intends to remove all in-breast neoplasia (both invasive cancer and ductal carcinoma in-situ, DCIS), to control the cancer locally and prevent its spread. In high-risk patients, with extensive disease within the breast, mastectomy is indicated.

### SUMMARY

According to a first aspect, a computer implemented method of segmenting a magnetic resonance (MR) image, comprises: segmenting a residual breast from an input MR image depicting the residual breast with implant after surgical removal of breast tissue, wherein the segmentation of the residual breast includes breast tissue remaining after the surgical removal, feeding the segmented residual breast into a fibroglandular tissue (FGT) segmentation ML model, and obtaining a segmentation of FGT from the segmented residual breast as an outcome of the FGT segmentation ML model.

In a further implementation form of the first aspect, the FGT segmentation ML model is trained on a FGT segmentation training dataset of a plurality of FGT segmentation records, each FGT segmentation record including a MR image of a segmented whole breast of a control subject that excludes an implant and on which no surgical removal of breast tissue has been performed, and a ground truth comprising a segmentation of the FGT depicted in the segmented whole breast.

In a further implementation form of the first aspect, the segmentation of the residual breast excludes the implant, and excludes surrounding non-breast tissue.

In a further implementation form of the first aspect, the MR image of the segmented whole breast of the FGT segmentation record is obtained by feeding an MR image of the whole breast into a whole breast segmentation ML model, wherein the whole breast segmentation ML model is trained on a whole breast segmentation training dataset of a plurality of whole breast segmentation records each including the MR image of the whole breast of the control subject that excludes the implant and on which no surgical removal of breast tissue has been performed, and a ground truth comprising a segmentation of the whole breast.

In a further implementation form of the first aspect, the segmentation of the FGT of the plurality of FGT segmentation records is performed by: co-registration between a first type of MR image of the whole breast of the control subject that excludes the implant and on which no surgical removal of breast tissue has been performed, and a second type of MR image of the whole breast of the control subject, the first type of MR image does not suppresses signals from fat tissue and the second type of MR image does suppress signals from fat tissue, applying a first threshold to the first MR image such that voxels with gray scale values below the first threshold to generate a first mask, applying a second threshold to the second MR image such that voxels with gray scale values above the second threshold to generate a second mask, labelling voxels that appear in the first mask and in the second mask as FGT.

In a further implementation form of the first aspect, the first type of MR image is a T2-weighted non-fat suppressed MR image or a T1-weighted non-fat suppressed MR image, and the second type of MR image is a T1-weighted fat suppressed MR image.

In a further implementation form of the first aspect, the first threshold denotes the lowest about 35-45% of the gray scale values, and the second threshold denotes the highest about 25-35% of the gray scale values.

In a further implementation form of the first aspect, segmenting the residual breast comprises segmenting the implant, and isolating residual breast from surrounding non-breast tissue and from the segmented implant.

In a further implementation form of the first aspect, isolating residual breast from the segmented implant comprises setting values of voxels of the segmented implant to zero.

In a further implementation form of the first aspect, segmenting the implant comprises segmenting the implant and capsule.

In a further implementation form of the first aspect, isolating the residual breast from surrounding non-breast tissue is performed by detecting an upper edge of the breast depicted in the input MR image, detecting a plurality of landmarks at a lower edge of the breast, and connecting between the plurality of landmarks.

In a further implementation form of the first aspect, the plurality of landmarks are detected by feeding the input MR image into a landmark ML model, wherein the landmark ML model is trained on a landmark training dataset of a plurality of landmark records, wherein a landmark record includes a sample MR image of a sample residual breast with implant and surrounding non-breast tissue, and a ground truth label marking of the plurality of landmarks.

In a further implementation form of the first aspect, segmenting the implant comprises feeding the input MR image into an implant segmentation ML model, wherein the implant segmentation ML model is trained on an implant segmentation training dataset of a plurality of implant segmentation records, wherein an implant segmentation record includes a sample MR image of a sample residual breast with implant after surgical removal of breast tissue, and a ground truth label segmentation of the implant.

In a further implementation form of the first aspect, in response to the implant being made of a synthetic material, segmenting the implant comprises: feeding the input MR image into a bounding box ML model to extract a bounding box around the synthetic material implant depicted therein, extracting the bounding box, and feeding the bounding box into a synthetic implant ML model that generates a segmentation of the synthetic material implant, wherein the bounding box ML model is trained on a bounding box training dataset of a plurality of bounding box records, wherein a bounding box record includes a sample MR image of an implant made of the synthetic material and after surgical removal of breast tissue, and a ground truth label of a bounding box enclosing the sample residual breast, wherein the synthetic implant ML model is trained on a training dataset of a plurality of synthetic implant records, wherein a synthetic implant record includes the bounding box of the bounding box record, and a ground truth segmentation of the synthetic implant.

In a further implementation form of the first aspect, the implant is made of autologous tissue.

In a further implementation form of the first aspect, further comprising computing at least one of: a volume of the segmented FGT, and a percentage of segmented FGT of a volume of the residual breast.

In a further implementation form of the first aspect, further comprising dividing the residual breast depicted in the input MR image into a plurality of regions, and computing for each region, at least one of: a volume of the segmented FGT, and a percentage of segmented FGT of a volume of the residual breast.

In a further implementation form of the first aspect, further comprising computing a risk score indicating likelihood of recurrence of breast cancer in the segmented FGT.

In a further implementation form of the first aspect, the risk score is computed by feeding the segmented FGT into a risk score ML model, wherein the risk score ML model is trained on a risk score training dataset of a plurality of risk score records, wherein a risk score record includes a segmented FGT of a sample subject and a ground truth label indicating recurrence of breast cancer in the segmented FGT of the sample subject.

In a further implementation form of the first aspect, further comprising treating the subject according to the segmented FGT with a treatment effective for preventing recurrence of the breast cancer, selected from a group comprising: radiation therapy, chemotherapy, and additional surgery.

In a further implementation form of the first aspect, the MR image comprises a plurality of 2D slices, wherein the input MR image comprises a single 2D slice of the plurality of 2D slices, and iterating the segmenting, the feeding, and the obtaining for each 2D slice of the plurality of 2D slices for obtaining a plurality of segmentations of FGT, and computing a volume and/or 3D segmentation of the FGT by aggregating the plurality of segmentations of FGT, wherein the plurality of 2D slices include at least 2D slice of each of: a central part of the implant with clear borders, including an edge of the implant with non-definitive borders, and no implant.

In a further implementation form of the first aspect, the input medical image is captured by a MR imaging device operating under a fat suppressed protocol.

In a further implementation form of the first aspect, further comprising feeding the segmented FGT into an image-based simulation process for planning radiation therapy for treating the FGT of subject.

In a further implementation form of the first aspect, further comprising: creating a second FGT segmentation training dataset of a plurality of second FGT segmentation records, each second FGT segmentation record including: a sample MR image of a sample subject depicting a sample residual breast with implant after surgical removal of breast tissue, and a ground truth comprising a segmented sample FGT, wherein the segmented sample FGT is segmented from the sample MR image by the segmenting the sample residual breast from the sample MR image, and segmenting the sample FGT from the segmented sample residual breast, and training a second FGT segmentation ML model on the second FGT segmentation training dataset, wherein the second FGT segmentation ML model generates a target segmented FGT in response to an input of a target MR image depicting a residual breast with implant after surgical removal of breast tissue.

In a further implementation form of the first aspect, segmenting the residual breast from the input MR image comprises: wherein the input MR image comprises a 3D volume, and further comprising: obtaining a plurality of 2D MR image slices representing the volume of the input MR image, feeding the plurality of 2D MR images slices as a single volume, into a 3D residual breast segmentation ML model, and obtaining the segmented residual breast for each of the plurality of 2D MR image slices as an outcome of the 3D residual breast segmentation ML model, wherein the 3D residual breast segmentation ML model is trained on a training dataset of a plurality of records, wherein a record includes a plurality of sample 2D MR images slices of a sample volume of a sample MR scan, and a ground truth indicating segmentation of the residual breast in each of the sample 2D MR images, wherein the 3D residual breast segmentation ML model is trained on the plurality of sample 2D MR image slices of the sample volume of the sample MR scan as a single volume.

In a further implementation form of the first aspect, the training dataset further includes a second set of FGT segmentation records, each record including an MR image of a residual breast on which surgery has been performed, with or without implant, and a ground truth comprising the segmentation of the FGT in the residual breast.

According to a second aspect, a computer implemented method for segmentation of FGT in a MR image, comprises: feeding an MR image depicting a whole breast of a target subject that excludes an implant and on which no surgical removal has been performed into a FGT segmentation ML model, and obtaining a segmentation of FGT of the MR image as an outcome of the FGT segmentation ML model, wherein the FGT segmentation ML model is trained on a FGT segmentation training dataset of a plurality of FGT segmentation records, each FGT segmentation record including an MR image of a segmented whole breast of a control subject that excludes an implant and on which no surgical removal has been performed, and a ground truth comprising a segmentation of the FGT depicted in the segmented whole breast, wherein the segmentation of the FGT is performed by: co-registration between a first type of MR image of the whole breast of the control subject, and a second type of MR image of the whole breast of the control subject, the first type of MR image does not suppresses signals from fat tissue and the second type of MR image does suppress signals from fat tissue, applying a first threshold to the first MR image such that voxels with gray scale values below the first threshold to generate a first mask, applying a second threshold to the second MR image such that voxels with gray scale values above the second threshold to generate a second mask, and labelling voxels that appear in the first mask and in the second mask as FGT.

According to a third aspect, a computer implemented method for training a FGT segmentation ML model for segmentation of FGT in a MR image, comprises: creating a FGT segmentation training dataset of a plurality of FGT segmentation records, each FGT segmentation record including an MR image of a segmented whole breast of a control subject that excludes an implant and on which no surgical removal has been performed, and a ground truth comprising a segmentation of the FGT depicted in the segmented whole breast, wherein the segmentation of the FGT is performed by: co-registration between a first type of MR image of the whole breast of the control subject, and a second type of MR image of the whole breast of the control subject, the first type of MR image does not suppresses signals from fat tissue and the second type of MR image does suppress signals from fat tissue, applying a first threshold to the first MR image such that voxels with gray scale values below the first threshold to generate a first mask, applying a second threshold to the second MR image such that voxels with gray scale values above the second threshold to generate a second mask, and labelling voxels that appear in the first mask and in the second mask as FGT, and training the FGT segmentation ML model on the FGT segmentation training dataset.

According to a fourth aspect, a computer implemented method of segmenting a MR image, comprises: for each 2D MR image slice of a plurality of 2D MR image slices depicting a volume of a subject that depicts one or two breasts: generating a segmented MR image by segmenting one or two breasts from surrounding non-breast tissue, dividing each segmented MR image into two sub-images each depicting a single breast or tissue remaining after mastectomy, classifying each sub-image into one of the following implant categories: breast with no implant, breast with synthetic implant, and breast with autologous implant, feeding each sub-image into one of a plurality of FGT segmentation ML models, each respective FGT segmentation ML model trained for a respective implant category, obtaining a segmentation of FGT as an outcome of the respective FGT segmentation ML model, and computing a volume and/or 3D segmentation of the FGT by aggregating the plurality of segmentations of FGT obtained for the plurality of 2D slices.

According to a fifth aspect, a computer implemented method of segmenting a MR image, comprises: segmenting a residual breast from an input MR image depicting the residual breast without implant and after surgical removal of breast tissue, wherein the segmentation of the residual breast includes breast tissue remaining after the surgical removal, and feeding the segmented residual breast into a fibroglandular tissue segmentation ML model, obtaining a segmentation of FGT from the segmented residual breast as an outcome of the FGT segmentation ML model, wherein the FGT segmentation ML model is trained on a FGT segmentation training dataset of a plurality of FGT segmentation records, each FGT segmentation record including an MR image of a segmented whole breast of a control subject that excludes an implant and on which no surgical removal of breast tissue has been performed, and a ground truth comprising a segmentation of the FGT depicted in the segmented whole breast.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a block diagram of components of a system 100 for segmentation of fibroglandular tissue (FGT) from an input MR image depicting residual breast tissue after surgical removal of breast tissue, in accordance with some embodiments of the present invention;

FIG. 2 is a flowchart of a method for segmentation of FGT from an input MR image depicting residual breast tissue after surgical removal of breast tissue, in accordance with some embodiments of the present invention;

FIG. 3A is a flowchart of an exemplary process for segmenting the residual breast from the MR image, in accordance with some embodiments of the present invention;

FIG. 3B is a flowchart of an exemplary process for training a FGT segmentation machine learning (ML) model for segmentation of FGT in a MR image, in accordance with some embodiments of the present invention;

FIG. 4 is an image depicting a detected upper edge of two breasts with implants, in accordance with some embodiments of the present invention;

FIG. 5 is an exemplary sample input MR image depicting ground truth segmentation, of an implant segmentation training dataset for training an implant segmentation ML model, in accordance with some embodiments of the present invention;

FIG. 6 is an image depicting a bounding box that encloses a right implant of a right breast and a bounding box that encloses a left implant of a left breast, in accordance with some embodiments of the present invention;

FIG. 7 includes an image depicting a residual breast tissue with an implant, and an image depicting a segmentation of the implant, in accordance with some embodiments of the present invention;

FIG. 8 is an example of a sample image of sample residual breasts with implants and surrounding non-breast tissue, and multiple landmarks, in accordance with some embodiments of the present invention;

FIG. 9 is an example of a sample image with a line connecting multiple landmarks, in accordance with some embodiments of the present invention;

FIG. 10 includes an example of a MR images depicting residual breasts remaining after surgical removal of breast tissue and implants, and an image depicting a segmentation of the residual breast of the MR image, in accordance with some embodiments of the present invention;

FIG. 11 includes an example of a sample MR image and a segmentation of a whole breast, in accordance with some embodiments of the present invention;

FIG. 12 is a sample MR image, and a FGT segmentation, in accordance with some embodiments of the present invention;

FIG. 13 is a graph of a leave-one-out cross validation for residual breast segmentation using a U-net architecture, in accordance with some embodiments of the present invention; and

FIG. 14 includes graphs for evaluation of a combination of two U-net architectures used for whole breast segmentation and FGT segmentation, in accordance with some embodiments of the present invention.

### DETAILED DESCRIPTION

The present invention, in some embodiments thereof, relates to MR images and, more specifically, but not exclusively, to systems and methods for analyzing MR images depicting residual breast tissue.

As used herein, the term MR image may sometimes refer to the MR imaging (MRI) scan which includes or is divided into multiple MR slices, or may sometimes refer to the individual MR slices of the MR scan. The MRI scan may be of the entire breast, while the MR slice is a thin cross section of a portion of the breast.

An aspect of some embodiments of the present invention relates to systems, methods, computing devices, and/or code (stored on a data storage device and executable by one or more processors) for segmentation of fibroglandular tissue (FGT) in an input MR image of a subject, optionally a MR image captured by a MRI machine, optionally following a fat suppression protocol such as fat suppressed T1-weighted MRI scan. The input MR image depicts a residual breast after surgical removal of breast tissue (i.e., mastectomy procedure), optionally with an implant. For clarity, a single breast is discussed, but it is to be understood that the input image may include two breasts, where sub-images of a single breast may be created by dividing the image with two breasts. Surgical removal may be, for example, mastectomy with/without reconstruction, for treatment of breast cancer and/or for prevention of breast cancer (e.g., in BRCA patients who undergo prophylactic mastectomy). A processor segments residual breast(s) tissue from the input MR image. The segmentation of the residual breast tissue includes breast tissue remaining after the surgery. The residual breast tissue may exclude the implant (where the input image depicts the implant). The residual breast excludes surrounding non-breast tissue, for example, bones (ribs, sternum), musculoskeletal chest wall, heart, lung, etc . . . The segmentation of the residual breast tissue may include a breast contour. The residual breast tissue includes FGT and may include other tissues of the breast such as one or more of: subcutaneous fat, glandular tissue, mammary glands, and other supportive cells such as stroma cells within the breast. The implant may be excluded from the segmentation by setting values of voxels of the segmented implant to zero and/or another value that is ignored by future processing and/or that indicates that the implant is excluded from the segmentation. The processor feeds the segmented residual breast tissue (that includes the optional breast contour) into a FGT segmentation machine learning (ML) model. A segmentation of FGT from the segmented residual breast is obtained as an outcome of the FGT segmentation ML model. Values may be computed for the segmentation of the FGT, for example, volume and/or distribution, optionally per region (e.g., per breast quarter), and/or risk of recurrence. The subject may be treated according to the segmentation of the FGT and/or according to the values, and/or treatment may be planned, for example, additional surgery, targeted radiotherapy, systemic therapy, and/or watch and wait.

The FGT segmentation ML model may be trained on a FGT segmentation training dataset of multiple FGT segmentation records. Each FGT segmentation record may include an MR image of a segmented whole breast of a control subject that excludes an implant and on which no surgical removal of breast tissue has been performed (e.g., healthy breasts), and a ground truth comprising a segmentation of the FGT depicted in the segmented whole breast. MR images of whole breasts and the ground truth of FGT segmented from the whole breasts are used for training the ML model for segmenting FGT from residual breasts, for example, for increasing performance of the FGT segmentation ML model (e.g., since whole breasts include a larger amount of FGT than residual breast tissue, enabling the FGT segmentation ML model to better learn to segment small amounts of FGT), and/or since segmented FGT from residual breasts may be difficult to obtain, and/or since segmented FGT from whole breasts may be automatically obtained as described herein. Optionally, the training dataset further includes a second set of FGT segmentation records that may include MR images of a residual breast on which surgery has been performed, with or without implant, and a ground truth comprising the segmentation of the FGT in the residual breast.

An aspect of some embodiments of the present invention relates to systems, methods, computing devices, and/or code (stored on a data storage device and executable by one or more processors) for training a FGT segmentation ML model for segmenting FGT tissue in an input MR image. The input MR image that is fed into the trained FGT segmentation ML model may be, for example, an image depicting a whole breast of a target subject that excludes an implant and on which no surgical removal has been performed, and/or an image depicting residual breast with implant after surgical removal of breast tissue. The processor creates a FGT segmentation training dataset that includes multiple FGT segmentation records. Each FGT segmentation record includes an image of a segmented whole breast of a control subject that excludes an implant and on which no surgical removal has been performed, and a ground truth of a segmentation of the FGT depicted in the segmented whole breast. The processor computes the segmentation of the FGT is by co-registration between a first type of image of the whole breast of the control subject, and a second type of image of the whole breast of the control subject. Registration may be performed, for example, using one or more transformations, such as similarity and/or translation. The first type of MR image does not suppresses signals from fat tissue, optionally, an MR image captured using a non-fat suppressed protocol, for example, a T2-weighted non-fat suppressed image and/or a T1-weighted non-fat suppressed image. The second type of image does suppress signals from fat tissue, optionally, an image captured using a fat suppressed protocol, for example, a T1-weighted fat suppressed image. The processor applies a first threshold to the first image such that voxels with gray scale values below the first threshold to generate a first mask, for example, the first threshold denotes the lowest about 35-45% of the gray scale values. The processor applies a second threshold to the second image such that voxels with gray scale values above the second threshold to generate a second mask, for example, the second threshold denotes the highest about 25-35% of the gray scale values. The processor labels voxels that appear in the first mask and in the second mask as FGT. The FGT segmentation ML model is trained on the FGT segmentation training dataset.

An aspect of some embodiments of the present invention relates to systems, methods, computing devices, and/or code (stored on a data storage device and executable by one or more processors) for computing a volume and/or segmentation of the FGT in multiple 2D slices of a subject, for example 2D slices that are of an MRI scan. The following processing is performed for each 2D slice that depicts one or two breasts: generating a segmented image by segmenting one or two breasts from surrounding non-breast tissue. Dividing each segmented image into two sub-images each depicting a single breast or tissue remaining after mastectomy. Classifying each sub-image into one of the following implant categories: breast with no implant, breast with synthetic implant, and breast with autologous implant. Feeding each sub-image into one of multiple FGT segmentation ML models, where each respective FGT segmentation ML model is trained for a respective implant category. And obtaining a segmentation of FGT as an outcome of the respective FGT segmentation ML model. The multiple segmentations of FGT obtained for the multiple 2D slices are aggregated, for example, for computing a volume and/or 3D segmentation of the FGT. The resulting volume and/or segmentation is for the FGT of the volume of the breast(s).

At least some embodiments described herein address the technical problem of automatically segmenting breast MR images with implant, and/or improve the technical field of image processing by automatically segmenting breast MR images with implant, and/or improve upon existing manual segmentation approaches.

Even with screening programs and new systemic therapies, mastectomy is still indicated in approximately 20% of the patients, who do not respond to primary systemic therapy. In the past decades the rate of mastectomy has increased, mostly as a result of refined surgical techniques that allow to resect the breast tissue while preserving the skin with/without the nipple and facilitate immediate breast reconstruction. These procedures inevitably leave residual breast tissue with the skin flap and below the nipple areola complex. This is often not easily noticed in physical examination. At least some embodiments described herein analyze MR images to identify breast tissue, including after skin sparing/nipple sparing mastectomy and implant-based reconstruction. This allows to identify high risk regions for residual breast cancer, or residual breast tissue that might develop a second breast cancer.

Breast MR images with an implant may be segmented differently that breast MR images without the implant. The 2D MR image slices of the breast including an implant(s) are heterogeneous, including slices with full implant, slices with partial implant, and slices without implant. The heterogeneous distribution of the 2D slices raises the following technical challenges that do not arise in breast MR images that exclude implants:
In slices that contain the central part (i.e., the full part) of the implant, the borders of the implant are usually clear and the volume of residual breast is relatively small. The small volume may be missed during processing by one or more of the ML models described herein, and/or one or more of the ML models described herein may identify the implant as residual breast.
In slices that contain the edges of the implant, the borders of the implant may be non-definitive. Residual breast volume may be larger. The identification of the implant by one or more of the ML models described herein may be less accurate than identification of implant in slices that contain the central part of the implant.
There may be slices with no implant at all.

Whether the slices depict the central part, the edges, or no implant, depends on the position of the slice. Some slices are from the central part of the implant and some slices are from the border of the implant. It is noted that the implant's volume is smaller than the breast. The difference between the slices is the area of the implant relative to the area of the breast slice. In slices depicting the center of the implant, the center of the implant takes up more area relative to the breast. In contrast, in slices obtained from the border of the implant, the implant is smaller relative to the breast. Moreover, the appearance of the implant may be different. It is noted that the implant is always full, never partial.

At least some implementations described herein address the technical problem of objectively, repeatedly, and/or accurately providing a segmentation of FGT in MR images of one or more breasts of a subject. The MR images may be of subjects after mastectomy and/or implant reconstruction. The segmentation may be of residual breast tissue that has remain after surgical removal of tissue (e.g., mastectomy) and in which an implant (e.g., synthetic, autologous) has been inserted. In other implementations, the MR images are of whole breasts of a subject that exclude the implant and/or on which no surgical removal of tissue has been performed. The technical problem may relate to computing a metric of the segmentation of FGT, for example, volume and/or distribution, optionally per region of the breast (e.g., quarter). An accurate quantification of the FGT of residual breast and/or composition thereof is used for the identification of high-risk areas for recurrence, for example, in order to tailor further treatment such as additional surgery and/or radiotherapy and improve disease survival and overall outcomes of breast cancer patients.

At least some implementations described herein improve the technical field of medical image processing, and/or the technical field of machine learning, by providing approaches for segmentation of FGT in MR images (e.g. residual breast tissue, breast with no implant that underwent surgical reconstruction, slices of breasts with implant where a specific slice does not depict the implant, and/or whole breasts with no implant and that did not undergo surgery), and/or computing a measurement of the segmented FGT (e.g., in residual breast tissue).

At least some implementations described herein improve over prior approaches of segmenting FGT in residual breast tissue, which is based on a user manually delineating the FGT in MR images and/or thresholding according to gray-values of pixels. Manual segmentation of FGT is highly dependent on expertise and is very time consuming, which makes it impractical to perform. Moreover, manual delineation of the FGT in residual breast tissue is inaccurate, for example, the surgical removal of breast tissue leaves behind a small amount of FGT. Segmentation based on thresholding is inaccurate, since thresholding may differ between images and/or subjects according to the contrast between the FGT and fat, which requires manual correction to improve accuracy of the results. Moreover, the FGT of the residual breast tissue is not uniform in shape and/or not a continuous region, and/or since the remaining amount of FGT is small, it is difficult to fully manually delineate all the FGT. The MRI scan may be 2D or 3D (the physics of acquisition is different). In both cases of 2D or 3D MRI scans, multiple 2D slices are obtained, by dividing the MRI scan, or directly by the scan. There may be many 2D slices per MR image scan (which may be a 3D scan, or multiple 2D slices scan), making it difficult and/or time consuming to manually delineate all FGT in all slices. The FGT may be difficult to visualize in order to delineate it, since it may appear very similar to implants located in the breast which are in proximity to the FGT of the residual breast tissue. Since manual segmentation of the FGT in residual breast tissue and/or segmentation based on thresholding is inaccurate, measurements of the segmented FGT are inaccurate. The difficulty in FGT manual delineation is mainly in cases where the FGT amount is low. The contrast of the FGT and implants may be similar. The main challenge is the possible proximity between FGT and implants which could challenge an accurate differentiation. Also, FGT may appear like very thin lines, sometimes similar to blood vessels, which are difficult to differentiate and difficult to manually delineate accurately.

Embodiments described herein for automatic segmentation of FGT in residual breast tissue are not merely an automation of a manual process, but introduce at least one feature which has no manual counterpart, for example, feeding MR images and/or segmentations into one or more trained machine learning models and/or training the ML model(s), and/or other automated approaches for segmentation of different portions of the image (e.g., breast contour, implant, capsule) as described herein.

At least some implementations described herein relate to computing a segmentation of FGT in MR images depicting one or more breasts of a subject. MR images of the breast are different than breast images captured by other modalities, for example, CT, ultrasound, and mammography. For example, to generate images: MR imaging uses strong magnetic fields, CT and mammography use X-rays, and ultrasound uses high frequency sound waves. In mammography, the breast is compressed, while in MR images the breast is not compressed. Furthermore, MR images provides much more information than image captured using other modalities. For example, an MR image includes more than a 100 (usually) 2D slices of the breast. MR imaging provides several contrast images (e.g., T1-weighted, T2-weighted) which may be used for radiology reading and may be used for analysis, as described herein. Moreover, segmentation approaches may be simpler on x-rays (e.g., mammography, CT images), using Hounsfield units (HU) or corresponding approaches for mammography images, but visibility of the breast glandular tissue is not good. Ultrasound is operator dependent, and therefore may generated different images by different operators. Yet another difference is that a volume of tissues, in particular of the breast, cannot be computed from mammography and/or ultrasound generated 2D images. In particular, volume cannot be computed when the breast is compressed as is performed for mammography. In mammography, the area of tissue of the breast can be computed relative to the area of the whole breast, but cannot be used to compute volume. Approaches designed to process breast images captured by other modalities (e.g., mammography, ultrasound) cannot be simply used or simply adapted to process MR images, for example, a process and/or machine learning model for segmentation of tissue in mammography images may not be operable for segmenting MR images. Approaches to process MR breast images cannot be simply derived from approaches to process breast images captured by other modalities (e.g., mammography, ultrasound).

At least some embodiments described herein address the aforementioned technical problem, and/or improve the aforementioned technical field, and/or improve upon existing manual approaches, by providing approaches for automatic segmentation of FGT in MR images. FGT in residual breasts appears differently than in whole breasts. For example, the volume of FGT in residual breasts is much smaller than in whole breasts, the distribution of FGT throughout the residual breast is different than the distribution of FGT in whole breasts, and in many cases the FGT in residual breasts is adjacent to the borders of the implant, which poses a technical challenge for segmentation of the FGT alone while excluding the implant. The MR image may depict a residual breast with implant and/or after surgical removal of breast tissue (e.g., mastectomy and/or reconstruction). The segmentation may be done by segmenting a residual breast from the MR image. The residual breast includes the breast tissue that remained after the surgical removal, which includes the FGT and may include subcutaneous fat. The residual breast excludes the implant. The residual breast excludes surrounding non-breast tissue (e.g., bones such as the ribs, sternum, musculoskeletal chest wall, heart, lung, etc . . . ). The segmented residual breast is fed into a FGT segmentation ML model. The segmentation of FGT is obtained from the FGT segmentation ML model. The FGT segmentation ML model is trained on a FGT segmentation training dataset of multiple FGT segmentation records. Each FGT segmentation record includes an MR image of a segmented whole breast of a control subject that excludes an implant and on which no surgical removal of breast tissue has been performed, and a ground truth comprising a segmentation of the FGT depicted in the segmented whole breast. The segmented FGT may be analyzed (e.g., volume and/or distribution of FGT is computed) and/or used for planning treatment (e.g., additional surgery, systemic therapy, radiotherapy) and/or for guiding administration of treatment (e.g., defining the target volumes for radiation therapy, defining different radiation dose according to risk). Alternatively, the MR image may depict a whole breast without implant and/or on which surgical removal of breast tissue has not been performed. The whole breast is segmented from the image. The whole breast excludes surrounding non-breast tissue. The segmentation of the whole breast is fed into the FGT segmentation ML model to obtain the segmented FGT.

At least some embodiments described herein automatically quantifies residual breast and glandular tissue volumes as well as its distribution, to provide accurate information to aid in medical decisions and/or treatment.

At least some embodiments described herein address the medical problem and/or improve the medical field, of radiation therapy in the setting of reconstruction, as radiation therapy is associated with 20-49% of complications including loss of the implant breast. Segmenting the FGT in the residual breast may be used for improved guidance of radiation therapy to reduce toxicity and improve disease control, thus widening the therapeutic window.

Additional details of the technical problem, technical field, and/or existing approaches are now described.

Breast cancer is the most common cancer in women worldwide. Removal of the breast (mastectomy) is one of the treatment options for non-metastatic breast cancer, however, local recurrences have been shown to occur in areas of residual breast tissue after mastectomy. However, in the past decade, the proportion of patients who undergo mastectomy and (immediate) breast reconstruction is increasing, even in patients who are eligible for breast conservation, as are patients who undergo bilateral mastectomy and breast reconstruction for unilateral breast cancer. Part of the reason for increasing rates of mastectomy is that mastectomy and reconstructive procedures have been refined over the past decades to deliver an aesthetic outcome as close as possible to that of the pre-mastectomy breast shape and contralateral intact breast. The popularity of breast augmentation surgeries with silicone implants in the healthy female population are assumed to be one of the factors associated with the acceptance of mastectomy with implant reconstruction. Even though new surgical procedures for mastectomy (nipple sparing mastectomy (NSM)/skin sparing mastectomy (SSM)) and breast reconstruction are considered “oncologically safe”, they were not evaluated in prospective randomized trials. Advances in these surgical techniques are important to patients and allow for immediate breast reconstruction.

At least some embodiments described herein address the medical problem of improving oncological outcomes (locoregional recurrence varies between 2-20% depending on disease stage, risk factors and treatment) and/or reduce potential radiation related toxicity that is high in case of reconstruction. The improved oncological outcomes are enabled by using the FGT which is segmented from residual breast tissue depicted in images, for example, by estimating of recurrence of cancer, defining the radiation target volumes and doses (e.g., radiation boost), indicating additional surgery, as described herein.

There are various amounts of breast glandular breast tissue after these types of mastectomies (skin sparing with/without nipple sparing), noted on imaging (mostly on MR images). The residual breast glandular tissue is in various amounts and locations, and is partly related to the surgeon expertise. Inventors mapped the spatial location of local breast cancer recurrence and performed a histopathological analysis of local recurrence and glandular tissue after skin sparing and nipple sparing mastectomy, to discover a link between FGT remaining after surgery and risk of cancer recurrence. Inventors evaluated a clinical cohort of BRCA mutation carriers after different types of breast surgery and showed that the cumulative incidences of ipsilateral breast cancer recurrence at 5 and 10 years were 9.8% and 27.4%, respectively, in patients who underwent mastectomy and reconstruction without radiation (non-postmastectomy radiation group) versus 2% and 11.3%, respectively, in the breast conservation plus radiation group (p=0.0183). None of the mastectomy patients who received radiation after mastectomy developed a local recurrence. However, radiation is currently not indicated for all mastectomy cases and may significantly increase the complications after breast reconstruction and even lead to implant loss. The cohort group in this study that did not receive radiation after mastectomy were very early disease stage without lymph node involvement, thus no indication for radiation. Inventors realized that an explanation for early local recurrences in this group was not the disease stage but rather the nature of the surgical procedure of skin sparing/nipple sparing that tends to leave more breast tissue behind, dermal lymphatics and potential residual tumor cells. Moreover, Inventors discovered that over 90% of all local recurrences after these procedures occur within five years, suggesting subclinical residual disease after the procedure (i.e., not diagnosed by available tools in clinical practice). At least some embodiments described herein address the aforementioned medical problem of reducing risk of recurrence of cancer in patients that underwent surgery and breast reconstruction, by automatically identifying residual breast FGT after mastectomy and/or reconstruction, which represent potential regions of recurrence of cancer and/or emergence of a new primary cancer. Currently, there are no clinical guidelines to perform such an evaluation after the procedure, and there are no tools that assist in postmastectomy imaging. In many countries, follow-up is done using physical examination only, thus there is no ability to assess residual breast tissue and recurrences are diagnosed by palpation.

Breast MR imaging is one of the key modalities for screening, diagnosis and follow up of breast cancer. In patients who were diagnosed with breast cancer, in the preoperative staging, breast MR imaging was found superior to other imaging modalities for tumor size estimation and detection of additional tumor foci in the ipsilateral and contralateral breast. The indications of breast MR imaging for screening are now revisited as growing evidence is suggesting that MR imaging depicts cancer lesions at an earlier stage than mammography. MR imaging is a highly sensitive tool for soft tissue, differentiating well between breast glandular tissue, fat tissue and other structures within the breast. Recent advances in breast MR imaging provide high in-plane resolution and high temporal resolution images with minimal artifacts and relatively short scan times. The T1-weighted dynamic contrast enhanced (DCE) is regarded the diagnostic scan and together with other sequences such as T2-weighted and diffusion-weighted allow for a multiparametric assessment of breast lesions. The sensitivity of breast MR imaging allows for an earlier detection of breast lesions (compared with mammography and ultrasound), an accurate characterization of the disease extent, contralateral disease, lymph node involvement and in many cases a highly informed discrimination between cancerous and benign lesions. It is also an advantageous imaging tool for the detection of breast cancer in patients with dense breasts. In addition, it is the preferred modality for the assessment of the response to primary systemic therapy (PST), therefore aids in treatment approach, including planning the type of surgery and radiation. At least some embodiments described herein are designed to analyze MR images.

Guidelines for standard clinical practice, breast MR imaging is not recommended for follow up after breast reconstruction, as it was not evaluated in clinical trials and there is no proof that it aids in prognosis, see for example, Expert Panel on Breast Imaging, Heller S L, Lourenco A P, Niell B L, Ajkay N, Brown A, Dibble E H, Didwania A D, Jochelson M S, Klein K A, Mehta T S, Pass H A, Stuckey A R, Swain M E, Tuscano D S, Moy L. ACR Appropriateness Criteria® Imaging After Mastectomy and Breast Reconstruction. J Am Coll Radiol. 2020, incorporated herein by reference in its entirety.

At least some embodiments described herein relate to approaches for automatically identifying and/or measuring (e.g., volume of) FGT that remained after mastectomy and reconstruction, where the remaining FGT represents a risk for recurrence of cancer.

At least some embodiments described herein relate to ML model(s) designed to receive a MR breast image as input, for identifying residual breast tissue (i.e., segment FGT), after mastectomy and breast reconstruction (artificial implant and/or autologous implant). The ML model(s) may be applied for example, for diagnostics (e.g., MR imaging done for follow-up after prophylactic mastectomy in BRCA carriers or after breast cancer) and/or for planning and/or applying further treatment (e.g., additional surgery if a large amount of glandular tissue associated with significant risk of recurrence is detected), and/or guide radiation therapy planning to volumes that are at high risk of recurrence, thus improving radiation outcome by improving local control and/or guide the radiation planning to reduce doses to low risk volume, thus potentially reducing toxicity from the radiation therapy.

At least some embodiments described herein generate automatic spatial identification of the volume of FGT, and/or a score for a high-risk FGT region (e.g., by training the ML model(s) on data from pretreatment MR imaging and/or post-surgery MR imaging).

Breast cancer is the most common cancer in females. Locoregional therapy, surgery and radiation aim to treat the cancer to control the disease locally and prevent its spread. Both treatments, and partly systemic therapy are guided by imaging to plan the approach. Breast surgery has progressed over the years to allow for better aesthetic outcomes. This includes a surgical resection of the breast glandular tissue while leaving the breast skin with/without nipple intact. The volume of the glandular breast tissue is replaced by implant-for example, silicon, tissue expander, and/or autologous implant from the patient's own body. Falsely, it is believed that this procedure resects all breast glandular tissue, thus reducing the risk of breast cancer recurrence to a minimum. However, it is inevitable that various amounts of breast glandular tissue (FGT) remain after surgery and often un-noted and might be a risk for early recurrence and/or a source of second breast cancer. This is devastating to patients that had their breast removed. Currently, there are no clinical guidelines to perform such an evaluation after the procedure, there are no tools that assist in postmastectomy imaging. In practice, some patients experience a recurrence of the cancer after mastectomy. Inventors discovered that these are high numbers compared to the published literature after the old types of mastectomies that do not preserve the breast skin. It is important for patients, and their quality of life to have the option for breast reconstruction immediately after surgery. In addition, most centers in the world provide these types of surgeries for breast cancer patients. At least some embodiments described herein provide an indication of the risk of cancer recurrence after such surgeries and/or assist in identifying areas of FGT that are at high risk of recurrence and/or at risk of a second primary.

The technical challenges of modern mastectomy and reconstruction procedures, which are aimed at achieving an aesthetic outcome, raise the chances for a relatively large amount of residual breast tissue including various amounts of glandular tissue. The residual breast tissue increases the chances for recurrence of breast cancer. At least some embodiments described herein provide accurate quantification of residual breast volume, glandular tissue volume and/or spatial location within the residual tissue, which may be used, for example, for medical decisions including preventive procedures to avoid recurrence.

At least some embodiments described herein improve upon prior approaches, and/or are different than prior approaches. For example, MR imaging-based algorithms for the quantification of breast and glandular tissue volumes in normal breasts (i.e., no mastectomy and reconstruction) have been developed, but cannot be used to quantify FGT in breasts in which tissue has been surgically removed and in which an implant (e.g., synthetic, autologous) has been placed, for example, due to the limited amount of residual FGT and/or distribution of residual FGT which is different than in whole breasts (without implant and/or that did not undergo surgery. The limited amount of residual FGT and/or distribution of residual FGT which is different in whole breasts is difficult to accurately identify. Moreover, presence of the implant, in particular an autologous implant, is difficult to accurately differentiate from the residual FGT. In contrast, at least some embodiments described herein are designed to segment FGT in MR images of breasts in which tissue has been surgically removed and in which an implant has been placed. In another example, a semi-quantification of residual breast after mastectomy and reconstruction has been shown by Zippel et al. based on manual measurements of the residual breast thickness at 4 pre-defined points in axial T2-weighted and sagittal T1-weighted MR images. However, these measurements are dependent on the radiologist's eye, are not necessarily accurate and do not consider the whole residual tissue but only several regions. In contrast, at least some embodiments described herein automatically and accurately segment the FGT in MR images.

Moreover, since the amount of residual breast is dependent on the type of surgery and the surgeon's expertise, an accurate quantification of the volume of the residual breast and/or a map of the glandular tissue within are used in order to calculate the risk for recurrence and/or take informed medical decisions which may include preventive procedures such as radiation therapy targeted to the glandular tissue regions (which has been shown to reduce recurrence in a BRCA cohort). Automatic quantifications of residual breast after mastectomy and reconstruction and quantification and mapping of the glandular tissue within have not been performed. At least some embodiments described herein provide an automatic, accurate, ML model-based determination of the residual breast volume and/or the glandular tissue volume that is present in the residual breast and its spatial location.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

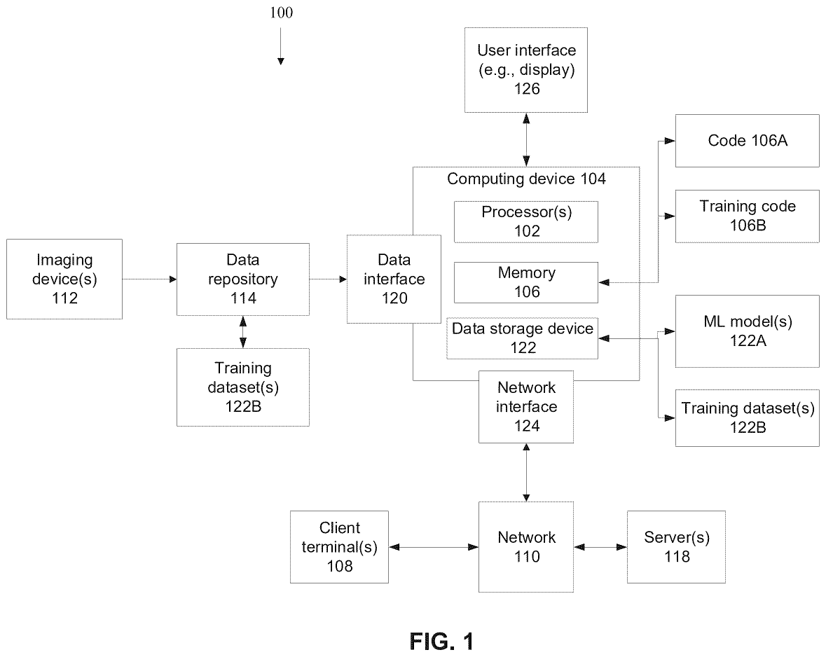

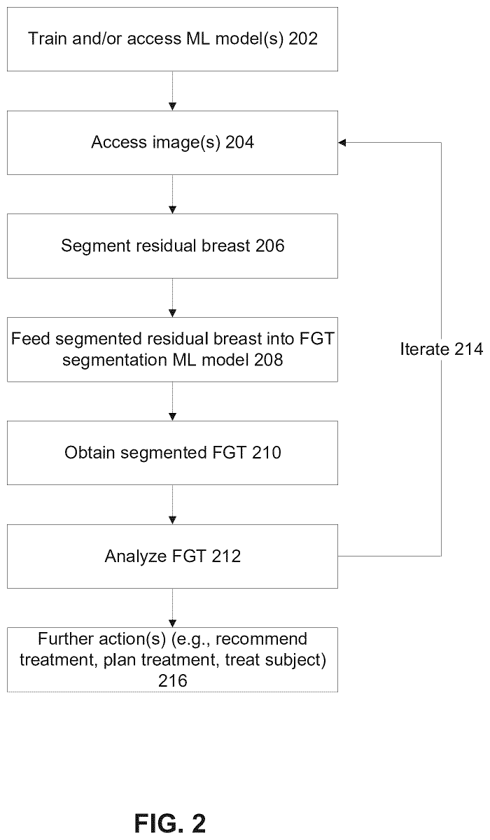

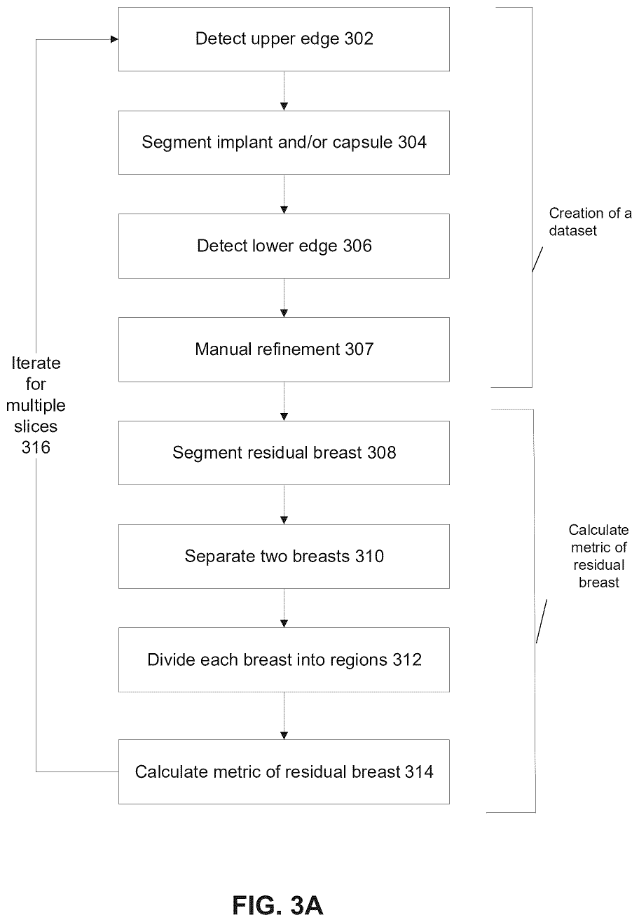

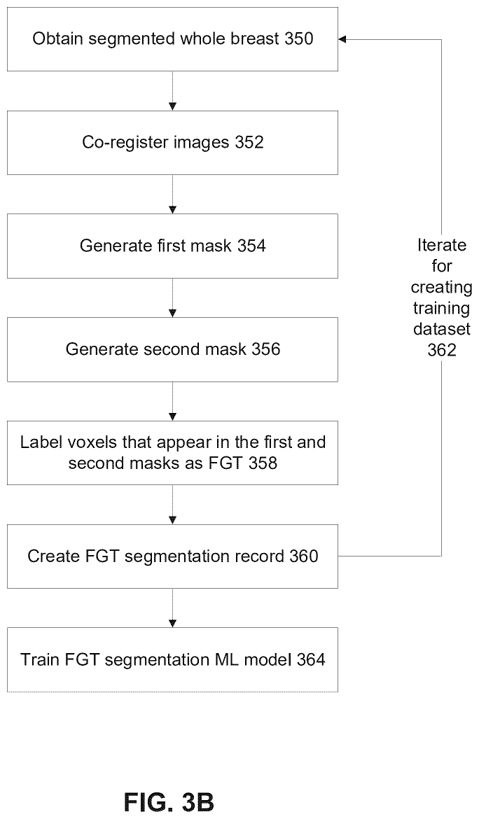

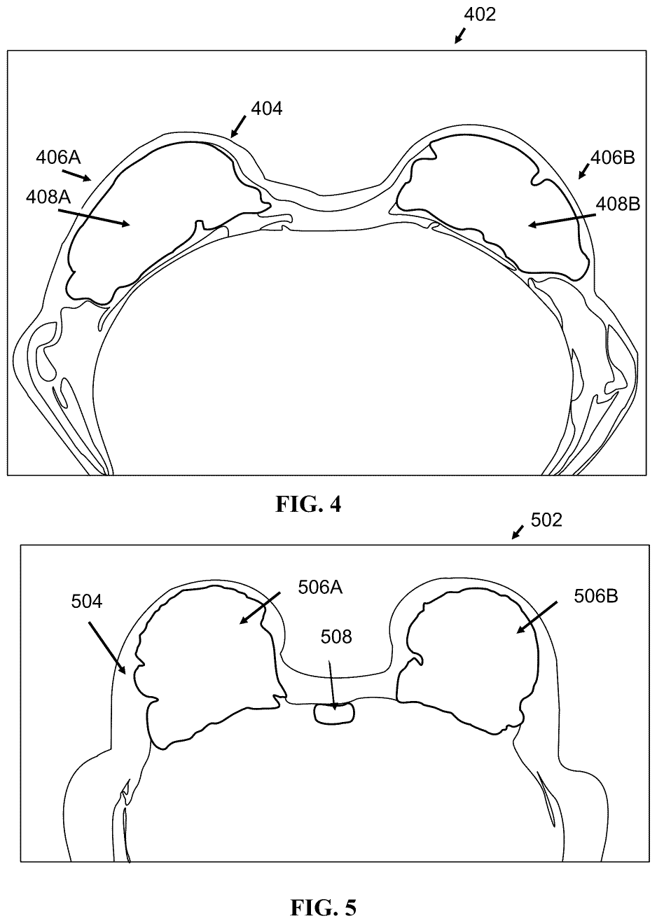

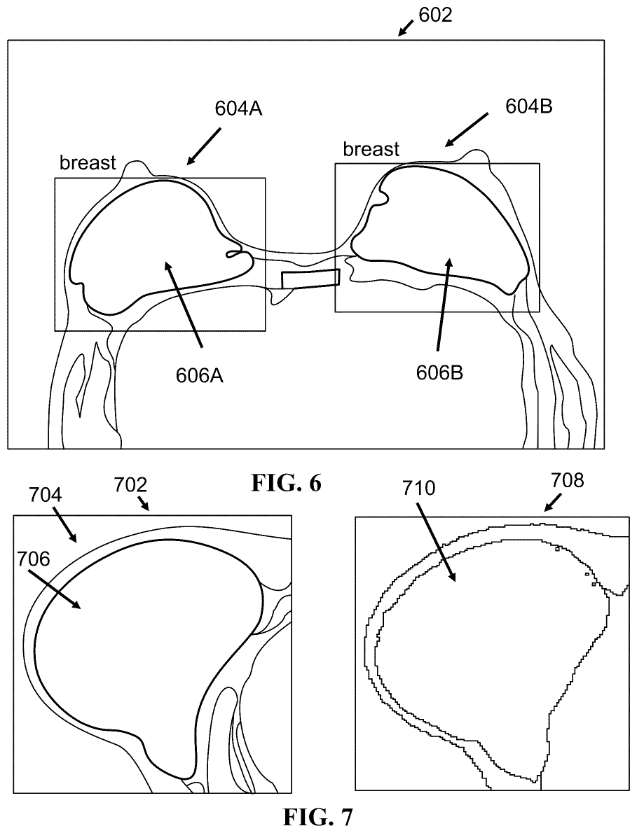

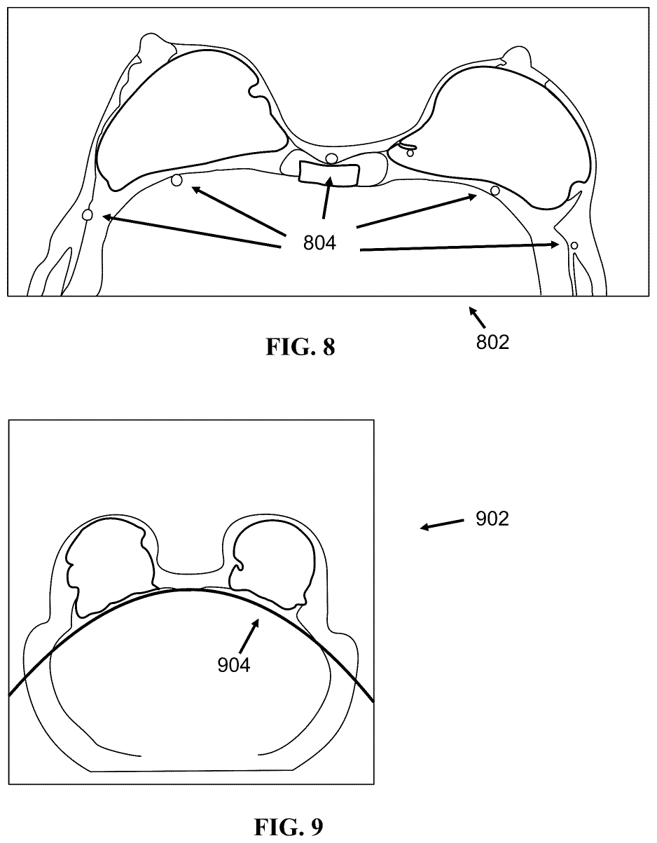

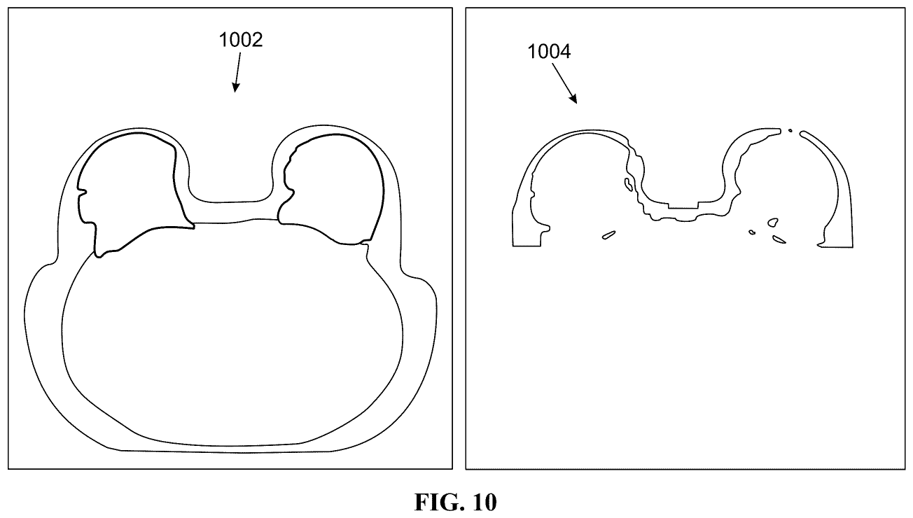

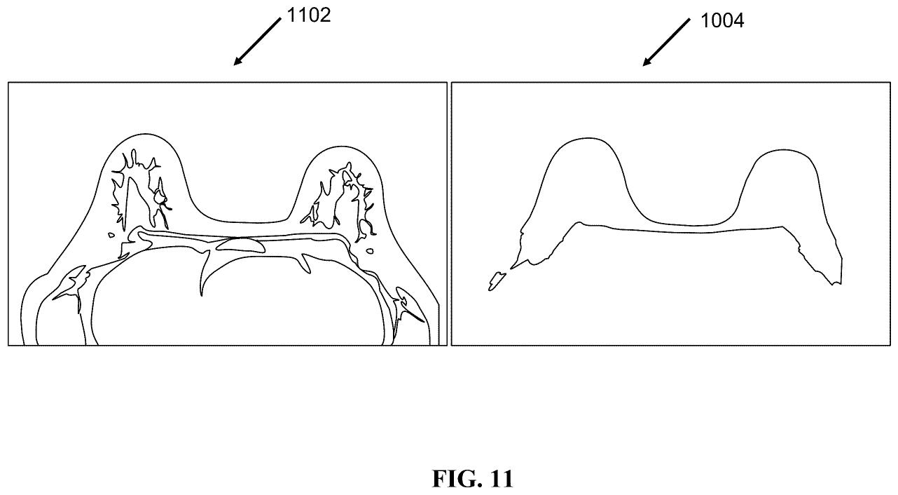

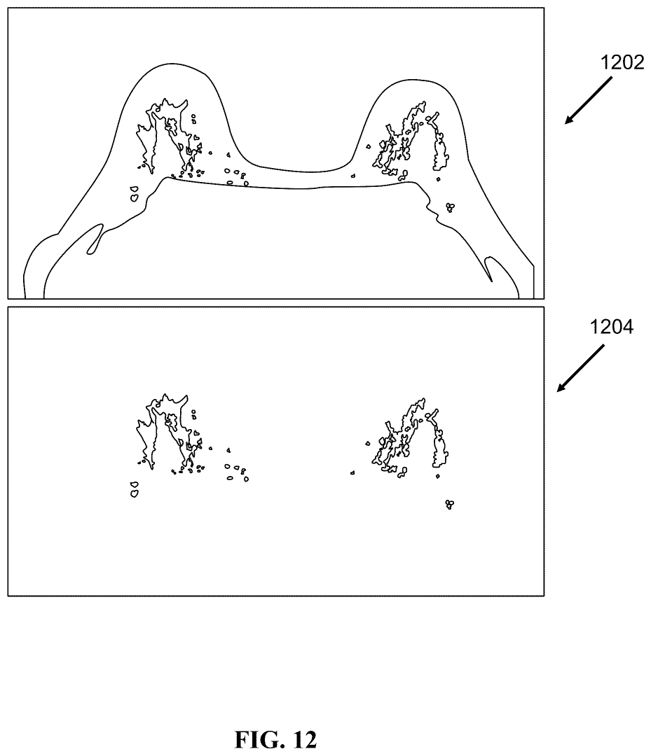

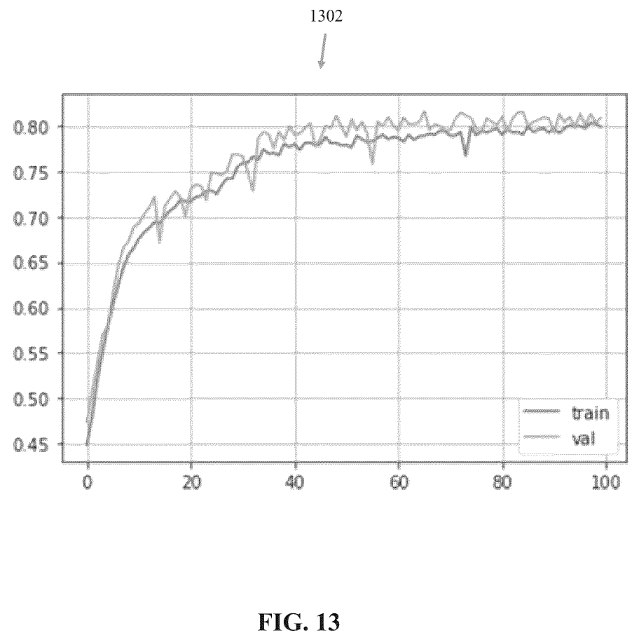

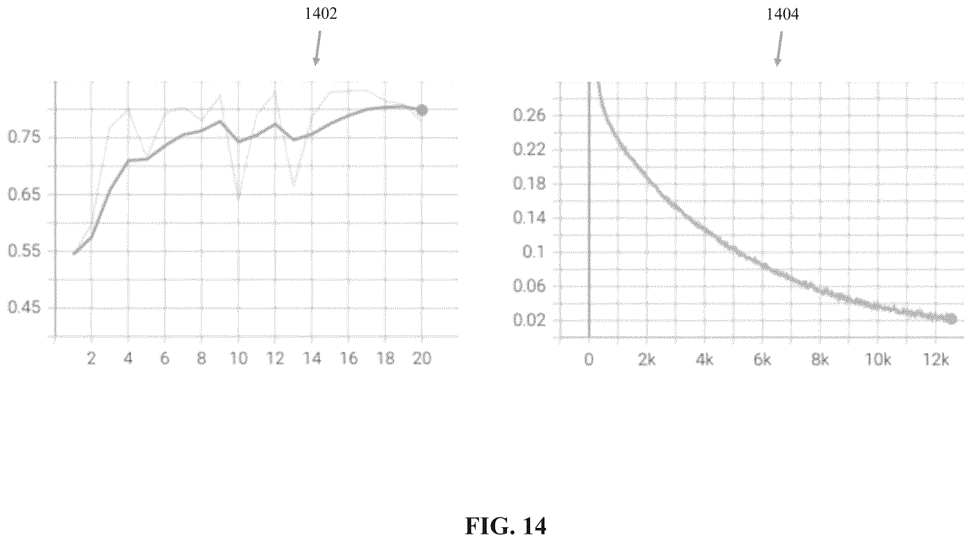

Reference is now made to FIG. 1, which is a block diagram of components of a system 100 for segmentation of FGT from an input MR image depicting residual breast tissue after surgical removal of breast tissue, in accordance with some embodiments of the present invention. Reference is also made to FIG. 2, which is a flowchart of a method for segmentation of FGT from an input MR image depicting residual breast tissue after surgical removal of breast tissue, in accordance with some embodiments of the present invention. Reference is also made to FIG. 3A, which is a flowchart of an exemplary process for segmenting the residual breast from the image, in accordance with some embodiments of the present invention. Reference is also made to FIG. 3B, which is a flowchart of an exemplary process for training a FGT segmentation ML model for segmentation of FGT in a MR image, in accordance with some embodiments of the present invention. Reference is now made to FIG. 4, which is an image 402 depicting a detected upper edge 404 of two breasts 406A 406B with implants 408 408B, in accordance with some embodiments of the present invention. Reference is also made to FIG. 5, which is an exemplary sample input MR image 502 depicting ground truth segmentation, of an implant segmentation training dataset for training an implant segmentation ML model, in accordance with some embodiments of the present invention. Reference is also made to FIG. 6, which is an image 602 depicting a bounding box 604A that encloses a right implant 606A of a right breast and a bounding box 604B that encloses a left implant 606B of a left breast, in accordance with some embodiments of the present invention. Reference is also made to FIG. 7, which includes an image 702 depicting residual breast tissue 704 with an implant 706, and an image 708 depicting a segmentation 710 of implant 706 of image 702, in accordance with some embodiments of the present invention. Reference is also made to FIG. 8, which is an example of a sample image 802 of sample residual breasts with implants and surrounding non-breast tissue, and multiple landmarks 804, in accordance with some embodiments of the present invention. Reference is also made to FIG. 9, which is an example of a sample image 902 with a line 904 (e.g., curve, set of lines connecting landmarks) connecting multiple landmarks, in accordance with some embodiments of the present invention. Reference is also made to FIG. 10, which includes an example of a MR image 1002 depicting residual breasts remaining after surgical removal of breast tissue and implants, and an image 1004 depicting a segmentation of the residual breast of the MR image 1002, in accordance with some embodiments of the present invention. Reference is also made to FIG. 11, which includes an example of a sample MR image 1102 and a segmentation of a whole breast 1104, in accordance with some embodiments of the present invention. Reference is also made to FIG. 12, which is a sample MR image 1202, and a FGT segmentation 1204, in accordance with some embodiments of the present invention. Reference is also made to FIG. 13, which is a graph 1302 of a leave-one-out cross validation for residual breast segmentation using a U-net architecture, in accordance with some embodiments of the present invention. Reference is also made to FIG. 14, which includes graphs 1402 and 1404 for evaluation of a combination of two U-net architectures used for whole breast segmentation and FGT segmentation, in accordance with some embodiments of the present invention.

System 100 may implement the acts of the method described with reference to FIGS. 2-14, optionally by a hardware processor(s) 102 of a computing device 104 executing code instructions stored in a memory 106.

Computing device 104 may be implemented as, for example, a client terminal, a server, a virtual server, a radiology workstation, a virtual machine, a computing cloud, a mobile device, a desktop computer, a thin client, a Smartphone, a Tablet computer, a laptop computer, a wearable computer, glasses computer, and a watch computer. Computing 104 may include an advanced visualization process that sometimes is add-on to a radiology workstation, for example, for presenting an image of the segmented residual breast tissue, computing a quantification of the residual breast tissue, and/or computing likelihood of recurrence of cancer in the residual breast tissue.

Computing device 104 may include locally stored software that performs one or more of the acts described with reference to FIGS. 2-14, and/or may act as one or more servers (e.g., network server, web server, a computing cloud, virtual server) that provides services (e.g., one or more of the acts described with reference to FIGS. 2-14) to one or more client terminals 108 (e.g., remotely radiology workstations, remote picture archiving and communication system (PACS) server, remote electronic medical record (EMR) server) over a network 110, for example, providing software as a service (Saas) to the client terminal(s) 108, providing an application for local download to the client terminal(s) 108, as an add-on to a web browser and/or a medical imaging viewer application, and/or providing functions using a remote access session to the client terminals 108, such as through a web browser.

Different architectures based on system 100 may be implemented.

In one example, computing device 104 provides centralized services. Computing device 104 may perform training of one or more ML models 122A, as described herein. Computing device 104 may analyze locally captured MR images (e.g., captured by imaging device 112) by feeding the MR images into ML model(s) 122A and/or other image processing approaches, as described herein. Alternatively, training is performed by another computing device, and inference is centrally performed by computing device 104. Locally captured MR images may be provided to computing device 104 for centralized analysis, for example, inference by the trained ML model(s) 122A. Images may be provided to computing device 104, for example, via an API, a local application, via a PACS server, and/or transmitted using a suitable transmission protocol. The outcome of the analysis (e.g., generation of segmentation of residual breast tissue, risk score for recurrence of cancer in the residual breast tissue, quantification of amount of residual breast tissue) may be provided, for example, to client terminal(s) 108 for presentation on a display and/or local storage, feeding into another process, stored in an electronic medical record (e.g., hosted by server 118), and/or stored by computing device 104. In another example, computing device 104 provides centralized training of the ML model(s) 122A, using records to create one or more training datasets 122B provided by different client terminals 108 and/or servers 118. For example, training datasets originating from different hospitals, and/or for different MR images (e.g., different settings of the MR imaging machine) and/or for different types of MR imaging machines (e.g., from different manufacturers) and/or for different cases such as different types of surgery and/or by different physicians. Respective generated ML models 122A may be provided to the corresponding remote devices (e.g., client terminal(s) 108 and/or server(s) 118) for local use. For example, each hospital uses the ML model created from their own training dataset for evaluation of new MR images captured at the respective hospital.

In another example, computing device 104 provides localized services. For example, computing device 104 includes code locally stored and/or locally executed by a radiology workstation, and/or client running a radiology image viewing program. The code may be a plug-in and/or add-on to the radiology image viewing program, to provide additional features of generating segmented images of residual breast tissue, computing risk scores for the residual breast tissue, and/or computing volumes/other metrics for the residual breast tissue. Computing device 104 may locally train ML model(s) 122A using training dataset(s) 122B of images captured by a local imaging device 112. In another example, computing device 104 obtains trained ML model(s) 122A from another device. Computing device 104 analysis MR images that are locally captured by imaging device 112, as described herein. The outcomes of the analysis may be presented on a display (e.g., user interface 126) of computing device 104, locally stored, sent to another device for storage (e.g., PACS server), and/or fed into another application (e.g., for guiding radiation therapy).

Imaging device 112 captures and provides the MR images, which may be included in training dataset(s) 122A and/or for inference. Imaging device 112 is a MRI machine that captures MR images. Multiple 2D slices may be obtained from an MR image (i.e., MR imaging scan).

Training dataset(s) 122B may be stored in a data repository 114 and/or data storage device 122, for example, a storage server, a computing cloud, virtual memory, and a hard disk. Training dataset(s) 122B are used to train the ML model(s) 122A, as described herein. It is noted that training dataset(s) 122B may be stored by a server 118, accessibly by computing device 104 over network 110.

Computing device 104 may receive images and/or records of the training dataset(s) 122B from imaging device 112 and/or data repository 114 using one or more data interfaces 120, for example, a wire connection (e.g., physical port), a wireless connection (e.g., antenna), a local bus, a port for connection of a data storage device, a network interface card, other physical interface implementations, and/or virtual interfaces (e.g., software interface, virtual private network (VPN) connection, application programming interface (API), software development kit (SDK)).

Hardware processor(s) 102 may be implemented, for example, as a central processing unit(s) (CPU), a graphics processing unit(s) (GPU), field programmable gate array(s) (FPGA), digital signal processor(s) (DSP), and application specific integrated circuit(s) (ASIC). Processor(s) 102 may include one or more processors (homogenous or heterogeneous), which may be arranged for parallel processing, as clusters and/or as one or more multi core processing units.

Memory 106 (also referred to herein as a program store, and/or data storage device) stores code instruction for execution by hardware processor(s) 102, for example, a random access memory (RAM), read-only memory (ROM), and/or a storage device, for example, non-volatile memory, magnetic media, semiconductor memory devices, hard drive, removable storage, and optical media (e.g., DVD, CD-ROM). For example, memory 106 may store image processing code 106A that implement one or more acts and/or features of the method described with reference to FIGS. 2-14 and/or training code 106B that trains one or more of the ML models described herein.

Computing device 104 may include a data storage device 122 for storing data, for example, one or more trained ML models 122A as described herein and/or one or more training datasets 122B as described herein. Data storage device 122 may be implemented as, for example, a memory, a local hard-drive, a removable storage device, an optical disk, a storage device, and/or as a remote server and/or computing cloud (e.g., accessed over network 110). It is noted that 122A-B may be stored in data storage device 122, with executing portions loaded into memory 106 for execution by processor(s) 102.

ML models 112A described herein (e.g., FGT segmentation ML model, second FGT segmentation ML model, landmark ML model, implant segmentation ML model, bounding box ML model, risk score ML model) may be implanted using a suitable architecture designed to process images, for example, one or more neural networks of various architectures (e.g., detector, convolutional, fully connected, deep, U-net, encoder-decoder, recurrent, graph, and combination of multiple architectures), for example, a 2D UNet, a 3D VNet, and the like.

Computing device 104 may include a network interface 124 for connecting to network 110, for example, one or more of, a network interface card, a wireless interface to connect to a wireless network, a physical interface for connecting to a cable for network connectivity, a virtual interface implemented in software, network communication software providing higher layers of network connectivity, and/or other implementations. Computing device 104 may access one or more remote servers 118 using network 110, for example, to obtain and/or provide training dataset(s) 116, an updated version of code 106A, training code 106B, and/or the trained ML model(s) 122A.

It is noted that data interface 120 and network interface 124 may exist as two independent interfaces (e.g., two network ports), as two virtual interfaces on a common physical interface (e.g., virtual networks on a common network port), and/or integrated into a single interface (e.g., network interface). Computing device 104 may communicate using network 110 (or another communication channel, such as through a direct link (e.g., cable, wireless) and/or indirect link (e.g., via an intermediary computing device such as a server, and/or via a storage device) with one or more of:
Client terminal(s) 108, for example, when computing device 104 acts as a server providing image analysis services (e.g., SaaS) to remote radiology terminals, for analyzing remotely obtained images using the trained ML model(s) 122A. Training dataset(s) 122B may be created based on images received from one or more client terminals 108.
Server 118, for example, implemented in association with a PACS, which may store image for training dataset(s) 122B and/or may store captured images for inference. Training dataset(s) 122B may be created from images stored by server 118.
MR imaging device 112 and/or data repository 114 that store images acquired by imaging device 112. The acquired images may be analyzed and/or fed into trained ML model(s) 122A for inference. Training dataset(s) 122B may be created based on images obtained from one or more imaging devices 112.

Computing device 104 and/or client terminal(s) 108 and/or server(s) 118 include and/or are in communication with a user interface(s) 126 that includes a mechanism designed for a user to enter data (e.g., manually define ground truth) and/or view the outcome of the analysis. Exemplary user interfaces 126 include, for example, one or more of, a touchscreen, a display, a keyboard, a mouse, and voice activated software using speakers and microphone.

Referring now back to FIG. 2, at 202, one or more ML models are provided and/or trained. Exemplary ML models and/or exemplary approaches to training the ML model(s) are provided herein.

Optionally, features described with reference to 204-214 (feature 212 may be excluded) are used to create a training dataset for training a second FGT segmentation ML model, in addition to the FGT segmentation ML model described herein. The second segmentation FGT ML model generates a target segmented FGT in response to an input of a target MR image depicting a residual breast with implant after surgical removal of breast tissue and/or in response to an input of a target MR image depicting a whole breast without implant and/or on which no surgery has been performed. The second segmentation FGT ML model may directly output the segmented FGT in response to the input of the input MR image. In contrast, the segmentation FGT ML model described herein outputs the segmented FGT in response to the input of the segmented residual breast (or segmented whole breast), where the segmented residual breast and/or whole breast is obtained from the input MR image. A second FGT segmentation training dataset of multiple second FGT segmentation records is created using features described with reference to 204-214. Each segmentation FGT record includes a sample MR image of a sample subject depicting a sample residual breast with implant after surgical removal of breast tissue and/or a whole breast without implant and/or on which no surgery has been performed (e.g. as described with reference to 204). A ground truth includes a segmented sample FGT which is segmented from the sample MR image by the segmenting the sample residual breast and/or whole breast from the sample MR image, and segmenting the sample FGT from the segmented sample residual breast and/or whole breast, as described with reference to 206-210. Multiple records may be created by iterating 204-210, as described with reference to 214. The second FGT segmentation ML model is trained on the segmentation FGT training dataset.

At 204, the processor accesses one or more input MR images (sometimes referred to as “image”).

The input MR image(s) may be one or more 2D slices of MR images (i.e., MRI scan). The MRI scan acquisition may be 2D or 3D (the physics of acquisition is different). In both cases of 2D or 3D MR imaging scans, multiple 2D slices are obtained, by dividing the MR imaging scan, or multiple 2D slices are directly acquired by the scan.

As used herein, the term MR image usually refers to the 2D slices, and sometimes refers to a single 2D slice. It is noted that each 2D MR image slice may be represented by voxels which represent a 3D volume, as opposed to pixels that represent a 2D area. As such, a volume of tissue, for example, the segmented FGT, may be computed by summing the volume of segmented FGT for each of the slices of a MR imaging scan.

Optionally, each input MR image depicts one or two residual breasts after surgical removal of breast tissue, for example, mastectomy and reconstruction. In some cases there is a single breast and/or a single implant, for example, where one breast has been removed and/or where a single implant has been inserted such as when a single breast underwent reconstruction. Each residual breast includes an implant, for example, made of synthetic material (e.g., silicon) and/or autologous tissue (e.g., fat and/or muscle obtained from another anatomical location on the subject's body). Optionally, the input MR image depicts a capsule in proximity to the residual breast tissue. The capsule is the border between the implant and the residual breast (e.g., the capsule looks like a black contour line around the implant on a non-fat suppressed MR image). The capsule is not a part of the residual breast, and therefore is removed. The capsule exists also in images with autologous implants (although less prominent) and is removed from image depicting autologous implants.

Alternatively or additionally, the input MR images depict whole breasts, without implant and/or on which no surgery has been performed.

Optionally, the input MR image(s) are captured by a MR imaging machine. The input MR images may be captured using one or more imaging protocols, for example, using a fat suppressed protocol, such as a fat suppressed T1-weighted MR imaging scan.

Breast MR imaging is one of the key modalities for screening, diagnosis and follow-up of breast cancer. Its relatively high spatial resolution and different possible contrasts allow for an accurate differentiation between the components of the breast including glandular tissue, fat, blood vessels and different kinds of lesions.

At 206, the processor segments the residual breast from the input MR image and/or segments the whole breast from the input MR image. The processor generates an image that depicts a breast contour and the residual breast tissue (or the whole breast).

When the input MR image depicts breast(s) with implant and tissue remaining after surgical removal (e.g., reconstruction), the segmentation of the residual breast includes breast tissue remaining after the surgical removal, including FGT and may include subcutaneous fat. The segmentation of the residual breast excludes the implant, and excludes surrounding non-breast tissue (e.g., bones (ribs, sternum), musculoskeletal chest wall, heart, lung, liver).

Optionally, the implant in the input MR image is segmented (e.g., as described herein). The implant may be excluded by setting values of voxels of the segmented implant to zero, and/or another value that is ignored by future processing (e.g., by the FGT segmentation ML model).

Alternatively, when the input MR image depicts whole breast(s) (i.e., without implant and/or on which no surgery has been performed, the segmentation of the residual breast includes the whole breast(s) and excludes the surrounding non-breast tissue.

An exemplary process for segmenting the residual breast from the input MR image is described below with reference to FIG. 3A.

Referring now back to FIG. 10, MR images 1002 (e.g., the input MR image) depicts residual breasts remaining after surgical removal of breast tissue and implants, and image 1004 depicts a segmentation of the residual breast of MR image 1002, which is segmented according to the features described with reference to 302-308 of FIG. 3A. MR image 1002 may a 2D MR slice of a MR imaging scan. The segmentation of the residual breast of MR image 1002 may be performed using a 3D ML model, for example, a 3D VNet model that may be trained on multiple 2D MR image slices as one volume. Alternatively, the residual breast is segmented by a 2D ML model trained on individual 2D MR images slices.

Referring now back to FIG. 13, graph 1302 depicts a leave-one-out cross validation performed by Inventors, for residual breast segmentation using a U-net architecture.

Referring now back to FIG. 2, at 206, optionally, the segmented image (of the residual breast or whole breast) is divided into two sub-images, for example, by detecting two non-continuous regions in the image (e.g., using image processing approaches and/or feeding into a separation ML model as described herein), and/or by identifying a central landmark of the multiple detected landmarks and drawing a vertical line, and/or halfway between the inner and/or outer edges of the breast contours, and/or halfway along the chest. The division is done such that each sub-image depicts a single breast or tissue remaining after mastectomy (i.e., where the breast used to be located prior to surgery). The division into sub-image may be done as described with reference to 310 of FIG. 3A.

At 208, the segmented residual breast (or the whole breast) is fed into a fibroglandular tissue segmentation ML model. The sub-image of a single residual breast (or single whole breast) may be fed into the FGT segmentation ML model.

The segmented residual breast that is fed into the FGT segmentation ML model may be a fat suppressed T1-weighted MR image.

The FGT segmentation ML model is trained on a FGT segmentation training dataset of multiple FGT segmentation records. Each FGT segmentation record includes an image of a segmented whole breast of a control subject that excludes an implant and on which no surgical removal of breast tissue has been performed, and a ground truth comprising a segmentation of the FGT depicted in the segmented whole breast.

One or more FGT segmentation records may include images of control subjects with one or more residual breasts that include one or more implants, and on which surgical removal of breast tissue has been performed and/or that underwent reconstruction. The ground truth includes the segmentation of the FGT depicted in the segmented residual breast, which may be obtained, for example, by manual segmentation. However, as described above, since it is difficult to obtain accurate manual segmentation of FGT in segmented residual breasts (e.g., is time consuming and/or requires an expert user to perform), such records may be in short supply. Moreover, the amount of FGT remaining in the residual breasts that underwent surgery may be small. As such, the number of records of segmented FGT is insufficient for training a FGT segmentation ML model to obtain a target performance (e.g., of a desired accuracy). The number of records of whole breasts and FGT segmentations may be large, and/or the amount of FGT in the whole breasts may be high, enabling training the FGT segmentation ML to obtain the target performance.

Optionally, the segmented image (of the residual breast or whole breast, optionally within each sub-image) is classified into one of the following implant categories: breast with no implant (e.g., whole breast), breast with synthetic implant, and breast with autologous implant. The classification may be done, for example, manually by a user, based on an analysis of the medical record of the user, and/or based on an analysis of the image (e.g., image analysis code that detects implants and/or detects the type of implant according to intensity values of the voxels, and/or feeding into a trained implant classifier trained on an implant training dataset of different images of breasts labelled with ground truth labels indicating respective classification categories). The segmented image is fed into one of multiple trained FGT segmentation ML models. Each respective FGT segmentation ML model is trained as described herein, using images with subjects of the respective implant category. The classification and selection of the corresponding type of FGT segmentation ML model may improve performance of the FGT segmentation ML model. It is noted that image slices of the same subject may differ in classification because the implant does not always fill in the whole volume of the breast, so some slices, especially in the extremities, may be classified as breast with no implant, while the rest of the slices will be classified as having an implant.

Alternatively, no classification is done, and the FGT segmentation ML model is trained on image depicting one or more (e.g. all) of the implant classification categories.

An exemplary process for segmenting the FGT from the whole breast and/or residual breast of the input MR image is described below with reference to 352-358 of FIG. 3B.

At 210, the processor obtains a segmentation of FGT (from the segmented residual breast and/or the segmented whole breast) as an outcome of the FGT segmentation ML model.

Referring now back to FIG. 12, sample MR image 1202 depicts whole breasts, without implants and that have not undergone surgery for removal of breast tissue. MR image 1202 may be, for example, a T1-weighted fat suppressed 2D slice of a MR image. FGT segmentation 1204 is obtained from sample MR image 1202, as described herein. FGT segmentation 1204 may be an outcome of the FGT segmentation ML model, and/or an example of a ground truth of a record of a training dataset for training the FGT segmentation ML model.

Referring now back to FIG. 2, at 212, the processor may analyze the segmented FGT.

Optionally, the processor computes a metric for the segmented FGT. Optionally, the metric is an area and/or a volume of the segmented FGT. Alternatively or additionally, the metric is a percentage of the segmented FGT from an area and/or volume of the residual breast.

The area of the segmented FGT may be computed based on the number of pixels depicting the residual breast and/or region, and the physical dimensions of tissue corresponding to each pixel.

Volume of the segmented FGT may be computed. The slices may be MR images that include voxels. The voxel may be defined as pixels with certain spatial resolution and slice thickness, defining a volume. Each voxel represents a certain volume, enabling computation of a volume of tissue, such as the segmented FGT, according to the volume of the voxels representing the tissue (e.g., segmented FGT). The volume of the segmented FGT may be computed by aggregating the volume of the segmented FGT computed for multiple 2D MR slices.

The area and/or volume of the residual breast may be computed, for example, as described with reference to feature 314 of FIG. 3A.

Alternatively or additionally, the processor may computing a risk score for the segmented FGT. The risk score may indicating likelihood of, for example, recurrence of breast cancer in the segmented FGT, a new second primary occurrence of cancer in the segmented FGT, a new primary cancer in the segmented FGT (e.g., for whole breasts without prior history of cancer), success of treatment in preventing cancer (e.g., where the treatment may be radiotherapy, surgery, chemo, and/or biological).

The risk score may be computed by feeding the segmented FGT into a risk score ML model. The risk score ML model may be trained on a risk score training dataset of multiple risk score records. A risk score record includes a segmented FGT of a sample subject (e.g., generated as descried herein) and a ground truth label indicating a sample risk score, for example, whether recurrence of breast cancer occurred in the segmented FGT of the sample subject, whether the subject experienced a second new occurrence of breast cancer, whether the subject experienced a new occurrence of breast cancer where there is no history of breast cancer, and/or whether administered treatment was effective.

The metric may be computed per region of the residual breast. Division into regions is described, for example, with reference to 312 of FIG. 3A. Alternatively or additionally, the metric is computed for an aggregation of the segmented FGT from multiple images (e.g., sequential slices), as described with reference to 214 of FIG. 2.

At 214, one or more features described with reference to 204-212 are iterated. The iterations may be performed for multiple sequential slices of the image, for example, 2D slices of a MR imaging scan of multiple 2D slices. The 2D slices may be directly obtained during the MR imaging scan, or may be obtained by dividing a 3D MR image into 2D slices.

The iterations may be performed for obtaining segmentations of FGT for each of the slices, and/or a volume and/or area of the FGT for each of the slices. A volume of the FGT of the breast(s), and/or a (e.g., 3D) segmentation of FGT of the breast(s) may be computed by aggregating the segmentations of FGT for each of the slices, and/or the volume and/or area of the FGT for each of the slices. The volume (and/or other metrics) of the segmented FGT may be computed, for example, as described with reference to 314 of FIG. 3A.

It is noted that breast MR images with and without an implant may be segmented differently. The 2D MR imaging slices of the breast depicting the implant may be heterogeneous:
In slices that contain the central part of the implant, the borders of the implant are usually clear and the volume of residual breast is relatively small. The small volume may be missed during processing by one or more of the ML models described herein, and/or one or more of the ML models described herein may identify the implant as residual breast.
In slices that contain the edges of the implant, the borders of the implant may be non-definitive. Residual breast volume may be larger. The identification of the implant by one or more of the ML models described herein may be less accurate than identification of implant in slices that contain the central part of the implant.
There may be slices with no implant at all.

At 216, one or more further actions may be taken in response to the segmented FGT, computed metrics, and/or risk scores. The actions may be manual (e.g., performed by physicians) and/or automatic (e.g., code generated by the processor). Examples of actions include:
Treating the subject according to the segmented FGT, computed metrics, and/or risk scores, for preventing or reducing likelihood of recurrence of breast cancer, for preventing or reducing likelihood of a second primary occurrence of breast cancer, and/or treating a recurrence of breast cancer, with an effective treatment. Exemplary treatments include: radiation therapy, additional surgery, chemotherapy, and biological therapy.
Watchful waiting. Taking no treatment action at the moment, and monitoring the subject by capturing another image at a future time.
Generating a recommendation for treating the subject.
Feeding the segmented FGT and/or computed metrics into an image-based simulation process for planning radiation therapy for treating the FGT of the subject. This enables identifying volumes and/or the spatial location of areas at risk of recurrence, and/or residual breast glandular tissue. This may allow for better dose coverage at volume of interest.
The segmentation of the FGT generated by at least some embodiments described may be used by a controller of radiation machines that incorporating MR imaging for planning and radiation delivery, for example, conformal radiotherapy.

Referring now back to FIG. 3A, an exemplary process for segmenting the residual breast from the input MR image is described. The exemplary process described with reference to FIG. 3A may be used for creating a dataset, which may be used for training, evaluating, and/or testing. Features described with reference to 302-307 may be for creation of the dataset. Features described with reference to 308-314 may be for calculation of a metric(s) (e.g., volume) of the residual breast.

At 302, the processor detects an upper edge of one or both breasts depicted in the input MR image, optionally the MR image slice. Optionally, a Wiener filter is applied to the input MR image. The upper edge is detected, for example, by an edge detection image processing processor that detects the edge based on a difference in pixel intensity values between the environment external to the body and tissues of the body.

Referring now back to FIG. 4, input MR image 402 depicts detected upper edge 404 of two breasts 406A 406B with implants 408 408B. Upper edge 404 is detected as described herein.

Referring now back to FIG. 3A, at 304, the processor segments the implant from the input MR image. The processor may segment the capsule.

Optionally, the implant is segmented by feeding the input MR image into an implant segmentation ML model. The implant segmentation ML model is trained on an implant segmentation training dataset that includes multiple implant segmentation records. An implant segmentation record includes a sample input MR image of a sample residual breast with implant after surgical removal of breast tissue, and a ground truth label segmentation of the implant.

Referring now back to FIG. 5, sample input MR image 502 is marked for indicating one or more ground truth labels, including: residual breast tissue 504, implants 506A and 506B, and sternum. Sample input MR image 502 is included in an implant segmentation record of an implant segmentation training dataset for training the segmentation ML model.

The ground truth labels may be generated, for example, by manual marking using an application such as 3D-slicer (e.g., available at www(dot)slicer(dor)org/), manual segmentation of each tissue type, and/or assignment of classification labels to each pixel according to the tissue type depicted by each pixel.

Implants 506A and/or 506B may be autologous implants and/or synthetic implants (e.g., made of silicon).

Optionally, when the implant is a synthetic implant, additional processing may be performed for segmentation of the synthetic implant. The additional processing may be done when the synthetic implant is visually distinguishable from the residual breast tissue. The visual distinction is due to the differences in appearance between human tissue and synthetic materials as depicted in the input MR image. Alternatively, the additional processing is performed regardless of whether the implant is made from synthetic material or is an autologous implant.

The additional processing may include the feature of feeding the input MR image into a bounding box ML model to extract a bounding box around the implant (e.g., synthetic implant, autologous implant) depicted therein. The processor extracts the bounding box from the input MR image. The processor feeds the bounding box into an implant ML model (e.g., synthetic implant ML model and/or autologous implant ML model) that generates a segmentation of the implant (e.g., synthetic material implant and/or autologous implant).

The bounding box ML model is trained on a bounding box training dataset that includes bounding box records. A bounding box record includes a sample image of a sample residual breast with implant (e.g., made of the synthetic material, and/or autologous implant) and after surgical removal of breast tissue, and a ground truth label of a bounding box enclosing the implant.

The bounding box ML model may be implemented as, for example, a R-CNN ResNet-5-FPN, or other architectures.

The (e.g., synthetic and/or autologous) implant ML model is trained on a training dataset of (e.g., synthetic and/or autologous) implant records. A synthetic and/or autologous implant record includes the bounding box of the bounding box record, and a ground truth segmentation of the synthetic implant and/or autologous implant.

The autologous and/or synthetic implant ML model may be implemented as, for example, a U-net architecture.

Referring now back to FIG. 6, image 602 depicts bounding box 604A that encloses right implant 606A of the right breast and bounding box 604B that encloses left implant 606B of the left breast. Image 602 may be an output of the bounding box ML model and/or may serve as a bounding box record of a bounding box training dataset for training the bounding box ML model.

Referring now back to FIG. 7, image 702 depicts residual breast tissue 704 with implant 706. Image 708 depicts a segmentation 710 of implant 706 of image 702. Images 702 and 708 may be part of an implant record of the implant training dataset for training the implant ML model. For example, image 702 represents the bounding box, and image 708 represents the ground truth segmentation. Alternatively or additionally, image 702 is an example input fed into the implant ML model, and image 708 is an example output of the implant ML model.

Referring now back to FIG. 3A, at 306, the processor detects a lower edge of the residual breasts in the input MR image.

The lower edge may be detected by detecting multiple landmarks on the chest wall that borders with at least one of: breast, implant, or residual breast (e.g., as depicted in FIG. 8). The landmarks may be connected, for example, by drawing lines between each pair of landmarks, fitting a curve to the landmarks, and the like.

The landmarks may be detected by feeding the input MR image into a landmark ML model. The landmark ML model may be trained on a landmark training dataset that includes landmark records. A landmark record includes a sample image of a sample residual breast with implant and surrounding non-breast tissue, and a ground truth label marking of the landmarks.

Alternatively or additionally, the landmark ML model is trained to generate one or more lines (e.g., curve) directly, without the intermediate step of outputting landmarks and then connecting the landmarks. In such embodiments, the landmark records include ground truth lines connecting the landmarks, and/or a curve(s) fitted to the landmarks.

The number of landmarks may be selected according to a desired accuracy for connecting the landmarks and/or for fitting a curve to the landmarks. For example, the number of landmarks may be 3, 4, 5, 6, 7, 8, or more.

Referring now back to FIG. 8, sample image 802 depicts sample residual breasts with implants and surrounding non-breast tissue, and multiple landmarks 804. Sample image 802 may be included in a landmark record of a landmark training dataset for training landmark ML model. Landmarks 804 are the ground truth labels. Landmarks 804 may be marked manually by a user. In another example, sample image 802 is fed into landmark ML model for generating landmarks 804 as an outcome thereof.

Referring now back to FIG. 9, sample image 902 includes one or more lines 904 connecting multiple landmarks (e.g., a curve fitted to the landmark). Line(s) 904 may be drawn by connecting pairs of landmarks automatically detected by the landmark ML model in response to an input of sample image 902. It is noted that landmarks are not visible in image 902, since line(s) 904 has been drawn to connect the landmarks.

At 307, manual refinement may be performed by a user, for example, by a radiologist specializing in breast imaging. Manual refinement may be performed, for example, via a GUI that presents the automated results and designed to enable the user to refine the automated results, for example, change the upper edge, lower edge, and/or segmentation boundaries. The manual refinement may be for features automatically detected as described with reference to 302-306.

Optionally, non-breast tissue is cleaned and/or excluded from the segmentation. The non-breast tissue may include one or more (e.g., all) of: between the two breasts (above the sternum), lateral to the breasts and axial slices that are non-breast (edge slices).

It is noted that features described with reference to 302-306 are in no particular order, and may be implemented, for example, in a different order (e.g., 302, 306, 304) and/or in parallel (e.g., 302 and 306 done in parallel followed by 304, or all of 302, 304, and 306 are done in parallel).

Features described with reference to 308-314 may be for calculation of a metric(s) (e.g., volume) of the residual breast.

At 308, the processor segments the residual breast from the MR image, optionally from the MR image slice. The segmentation may be performed automatically by a residual breast segmentation ML model trained on MR image slices marked with ground truth of the residual breast. The residual breast segmentation ML model may be implemented as a 3D model that is fed the 2D MR image slices, optionally multiple 2D MR slices of the MR imaging scan as a single volume. The 3D version of the residual breast segmentation ML model may be implemented as, for example, a 3D Vnet model. The 3D version of the residual breast segmentation MI model may be trained on multiple sample 2D slices of a sample MR imaging scan treated as a single volume, marked with the ground truth of the segmented residual breast in each of the 2D slices. The 3D residual breast segmentation ML model may output a segmentation of the residual breast on each of the input MR image slices which were fed as a single volume. The 3D version may be applied for multiple slices together as a single volume which considers continuity between slices, in comparison to a 2D version of the segmentation ML model applied per slice and therefore doesn't consider the continuity between slices. The 2D version of the residual breast segmentation ML model may be trained on sample 2D MR image slices marked with a ground truth of segmentation of the residual breast.

Examples of the MR image (e.g., 3D) 1002 and segmentation 1004 thereof are described with reference to FIG. 10.

The breast(s) is defined between the upper edge and the line(s) that defines the lower edge. The processor isolates the breast(s) from the surrounding non-breast tissue that is above the upper edge identified as described with respect to 302 and/or non-breast tissue that is below the identified lower edge as described with reference to 306.

Between the upper and lower edges there is residual breast tissue, optional capsule, and implant.

The lower and upper edges may be either along the residual breast or the implant depending on the breast slice.

The residual breast is segmented from the segmented implant (as described with reference to 304) and/or from the capsule.

At 310, in MR images that depict two residual breasts, the two breasts may be separated. Separation of the two residual breasts enables, for example, computing metrics and/or risk scores for per breast.

The processor may detect two non-continuous regions in the image. The two non-continuous regions represent the two residual breasts. The two non-continuous regions may be detected, for example, by image processing approaches (e.g., edge detection, feature extraction) and/or by predicting the two non-continuous regions by feeding the image into a separation ML model. The separation ML model may be trained on a training dataset of records, where a record may include an MR image depicting two residual breasts, and a ground truth indicating the two non-continuous regions (e.g., marked by a user). Alternatively or additionally the processor may compute a vertical line between the two residual breasts, for example, the vertical line is placed according to a middle landmark of multiple detected landmarks, and/or the vertical line is placed mid-way between two identified boundary boxes and/or between the two identified residual breasts.

The division of the image into sub-images, each including a single breast (or residual breast) may be done on the segmentation of the residual breast, on the original input MR image (e.g., 2D slice), and/or at a different stage in the process.

At 312, optionally, each breast (e.g., divided as described with reference to 310) is divided into regions, for example, halves, thirds, quarters, and the like. The division may be done based on the detected boundary boxes, for example, the are depicted in each boundary box is divided into multiple regions. The division into region enables, for example, computing metrics and/or risk scores per region.

At 314, one or more metrics may be computed per residual breast and/or per region, for example, the metrics (e.g., volume) may be computed for the segmented FGT. The area and/or volume of the residual breast and/or region and/or segmented FGT may be computed. The area may be computed based on the number of pixels depicting the residual breast and/or region and/or FGT, and the physical dimensions of tissue corresponding to each pixel. Volume may be computed based on the voxels of the MR slice. Each 2D MR image slices may include spatial resolution and slice thickness, where voxels represent the slice thickness or portion thereof. Each voxel represents a certain volume, enabling computation of a volume of tissue represented by multiple voxels. Alternatively, volume may be computed as the area computed for 2D slices (e.g., from 2D pixels) multiplied by the thickness of each slice. Alternatively or additionally, the metrics for the residual breast and/or per region of the residual breast may be performed in 212 of FIG. 2.

At 316, features described with reference to 302-314 may be iterated over multiple MR image slices of the MR scan (where each iteration is for one slice). The metrics may be computed for a volume by aggregating the results of the multiple slices.

Referring now back to FIG. 3B, an exemplary process for training the FGT segmentation ML model for segmentation of FGT in a MR image, is now described. The process provides an automated approach for segmentation of FGT in images of whole breasts. Alternatively or additionally, the process described herein with reference to FIG. 3B may be implemented on images of segmented residual breasts, which were segmented, for example, as described with reference to FIG. 3A, in particular 302-307 which relate to creation of a dataset for training and/or evaluation and/or testing. Features described with reference to 350-362 performed on images of whole breasts may alternatively or additionally be performed on images of segmentation of residual breasts.

At 350, an image of a segmented whole breast of a control subject that excludes an implant an on which no surgical removal has been performed, is obtained. The segmentation of the whole breast excludes non-surrounding tissues (e.g., chest wall, heart, lungs, liver).

The image of the segmented whole breast may be obtained by feeding an image of the whole breast into a whole breast segmentation ML model. The whole breast segmentation ML model may be trained on a whole breast segmentation training dataset of multiple whole breast segmentation records. Each record includes the image of the whole breast of the control subject that excludes the implant and on which no surgical removal of breast tissue has been performed, and a ground truth comprising a segmentation of the whole breast. Alternatively or additionally, the image of the segmented whole breast may be obtained by identifying the upper edge (e.g., as described with reference to 302 of FIG. 3A), lower edge (e.g., as described with reference to 306 of FIG. 3A), optional manual refinement (e.g., as described with reference to 307 of FIG. 3A) and/or as described with reference to 308 of FIG. 3A. Alternatively or additionally, a segmentation of residual breast is obtained from an image of a breast that includes an implant and/or that underwent surgery.

Alternatively or additionally, images of segmented residual breasts may be used. Residual breasts may be segmented from images depicting the residual breasts after surgery, with optional implant, as described herein.

Optionally, a heterogeneous composition of different breast slices of a patient are used. The breast may appear differently in the different slices, for example, even where there is an implant, some edge slices may not depict the implant. Images are obtained from different subjects, for example, one or more of: subjects with whole breasts that did not undergo surgical reconstruction, subjects that did undergo surgical reconstruction, subjects with residual breast without implant, subjects with residual breast with implant, and/or subjects with muscle and residual breast. Using a wide range of slices and/or different subjects may enable the ML model to learn to segment FGT in different cases (e.g., to account for different surgical procedures, different surgeons, and/or different reconstructions, and/or different implant states), providing high performance.

Referring now back to FIG. 11, sample MR image 1102 depicts whole breasts, without implants and that have not undergone surgery for removal of breast tissue. MR image 1102 may be, for example, a T2-weighted non-fat suppressed 2D slice of a MR image. Segmentation of whole breasts 1104 is obtained from sample MR image 1102, as described herein. Segmentation of whole breasts 1104 is performed, as described herein. The segmentation of the whole breasts 1104 may be fed into the FGT segmentation ML model, and/or FGT may be segmented from the segmentation of the whole breasts 1104 for creating the FGT segmentation training dataset for training the FGT segmentation ML model, as described herein.

Referring now back to FIG. 14, graphs 1402 and 1404 are an evaluation performed by Inventors, of the combination of two U-net architectures used for whole breast segmentation and FGT segmentation. Whole breast segmentation was based on about 6000 breast MR imaging slices and FGT segmentation was based on about 2500 slices. Graph 1402 is a dice coefficient change graph. Graph 1404 is a training loss graph.

Referring now back to 350 of FIG. 3B, segmentation of the FGT is described with reference to 352-358.

At 352, a first type of MR image of the whole breast (and/or segmented residual breast) of the control subject is co-registered with and a second type of MR image of the whole breast of the control subject. The first type of MR image does not suppresses signals from fat tissue, for example, the first type of MR image is a T2-weighted non-fat suppressed MR image or a T1-weighted non-fat suppressed image. The second type of MR image does suppress signals from fat tissue, for example, the second type of MR image is a T1-weighted fat suppressed MR image.

At 354, a first mask is generated by applying a first threshold to the first image, such that voxels with gray scale values below the first threshold are included in the first mask. The first threshold denotes the lowest of about 35-45%, or about 37-43%, or about 40% of the gray scale values, or other values. The specific percentage used for the threshold may vary between subjects and/or images.

At 356, a second mask is generated by applying a second threshold to the second image, such that voxels with gray scale values above the second threshold are included in the second mask. The second threshold denotes the highest of about 25-35%, or about 27-33%, or about 30% of the gray scale values, or other values.

At 358, voxels that appear in the first mask and in the second mask are labelled as FGT. The segmentation of the FGT is defined as the voxels labelled as FGT.

At 360, a FGT segmentation record is created. The FGT segmentation record includes the image of the segmented whole breast (and/or segmented residual breast) obtained as described with reference to 350, and a ground truth of the segmentation of the FGT depicted in the segmented whole breast (and/or segmented residual breast), obtained as the labelled FGT voxels described with reference to 358.

At 362, features described with reference to 350-360 are iterated, for creating multiple FGT segmentation records. A FGT segmentation training dataset that includes multiple FGT segmentation records is created.

The iterations may be performed for multiple slices of multiple MR scans, where each iteration may be for one slice of a certain MR scan. Each MR scan is associated with multiple slices.

Optionally, the training dataset further includes a second set of FGT segmentation records that may include MR images of a residual breast on which surgery has been performed, with or without implant, and a ground truth comprising the segmentation of the FGT in the residual breast. The second set of FGT segmentation records may be created as described with reference to 350-362, where in 350, a residual breast is substituted for the whole breast.

At 364, the FGT segmentation ML model is trained on the FGT segmentation training dataset.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

It is expected that during the life of a patent maturing from this application many relevant ML models will be developed and the scope of the term ML model is intended to include all such new technologies a priori.

As used herein the term “about” refers to ±10%.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A computer implemented method of segmenting a magnetic resonance (MR) image, comprising:
segmenting a residual breast from an input MR image depicting the residual breast with implant after surgical removal of breast tissue,
wherein the segmentation of the residual breast includes breast tissue remaining after the surgical removal;
feeding the segmented residual breast into a fibroglandular tissue (FGT) segmentation ML model; and
obtaining a segmentation of FGT from the segmented residual breast as an outcome of the FGT segmentation ML model.

2. (canceled)

3. The computer implemented method of claim 1, wherein the segmentation of the residual breast excludes the implant, and excludes surrounding non-breast tissue.

4. The computer implemented method of claim 1, wherein the MR image of the segmented whole breast of the FGT segmentation record is obtained by feeding an MR image of the whole breast into a whole breast segmentation ML model,
wherein the whole breast segmentation ML model is trained on a whole breast segmentation training dataset of a plurality of whole breast segmentation records each including the MR image of the whole breast of the control subject that excludes the implant and on which no surgical removal of breast tissue has been performed, and a ground truth comprising a segmentation of the whole breast.

5-7. (canceled)

8. The computer implemented method of claim 1, wherein segmenting the residual breast comprises segmenting the implant, and isolating residual breast from surrounding non-breast tissue and from the segmented implant.

9. (canceled)

10. The computer implemented method of claim 8, wherein segmenting the implant comprises segmenting the implant and capsule.

11. The computer implemented method of claim 8, wherein isolating the residual breast from surrounding non-breast tissue is performed by detecting an upper edge of the breast depicted in the input MR image, detecting a plurality of landmarks at a lower edge of the breast, and connecting between the plurality of landmarks.

12. The computer implemented method of claim 11, wherein the plurality of landmarks are detected by feeding the input MR image into a landmark ML model,
wherein the landmark ML model is trained on a landmark training dataset of a plurality of landmark records, wherein a landmark record includes a sample MR image of a sample residual breast with implant and surrounding non-breast tissue, and a ground truth label marking of the plurality of landmarks.

13. The computer implemented method of claim 8, wherein segmenting the implant comprises feeding the input MR image into an implant segmentation ML model, wherein the implant segmentation ML model is trained on an implant segmentation training dataset of a plurality of implant segmentation records, wherein an implant segmentation record includes a sample MR image of a sample residual breast with implant after surgical removal of breast tissue, and a ground truth label segmentation of the implant.

14. The computer implemented method of claim 13, wherein in response to the implant being made of a synthetic material, segmenting the implant comprises:
feeding the input MR image into a bounding box ML model to extract a bounding box around the synthetic material implant depicted therein,
extracting the bounding box, and
feeding the bounding box into a synthetic implant ML model that generates a segmentation of the synthetic material implant,
wherein the bounding box ML model is trained on a bounding box training dataset of a plurality of bounding box records, wherein a bounding box record includes a sample MR image of an implant made of the synthetic material and after surgical removal of breast tissue, and a ground truth label of a bounding box enclosing the sample residual breast,
wherein the synthetic implant ML model is trained on a training dataset of a plurality of synthetic implant records, wherein a synthetic implant record includes the bounding box of the bounding box record, and a ground truth segmentation of the synthetic implant.

15. (canceled)

16. The computer implemented method of claim 1, further comprising computing at least one of: a volume of the segmented FGT, and a percentage of segmented FGT of a volume of the residual breast.

17. The computer implemented method of claim 1, further comprising dividing the residual breast depicted in the input MR image into a plurality of regions, and computing for each region, at least one of: a volume of the segmented FGT, and a percentage of segmented FGT of a volume of the residual breast.

18. The computer implemented method of claim 1, further comprising computing a risk score indicating likelihood of recurrence of breast cancer in the segmented FGT.

19-20. (canceled)

21. The computer implemented method of claim 1, wherein the MR image comprises a plurality of 2D slices, wherein the input MR image comprises a single 2D slice of the plurality of 2D slices, and iterating the segmenting, the feeding, and the obtaining for each 2D slice of the plurality of 2D slices for obtaining a plurality of segmentations of FGT, and computing a volume and/or 3D segmentation of the FGT by aggregating the plurality of segmentations of FGT, wherein the plurality of 2D slices include at least 2D slice of each of: a central part of the implant with clear borders, including an edge of the implant with non-definitive borders, and no implant.

22. The computer implemented method of claim 1, wherein the input medical image is captured by a MR imaging device operating under a fat suppressed protocol.

23-24. (canceled)

25. The computer implemented method of claim 1, wherein segmenting the residual breast from the input MR image comprises:
wherein the input MR image comprises a 3D volume, and further comprising:
obtaining a plurality of 2D MR image slices representing the volume of the input MR image;
feeding the plurality of 2D MR images slices as a single volume, into a 3D residual breast segmentation ML model; and
obtaining the segmented residual breast for each of the plurality of 2D MR image slices as an outcome of the 3D residual breast segmentation ML model,
wherein the 3D residual breast segmentation ML model is trained on a training dataset of a plurality of records, wherein a record includes a plurality of sample 2D MR images slices of a sample volume of a sample MR scan, and a ground truth indicating segmentation of the residual breast in each of the sample 2D MR images, wherein the 3D residual breast segmentation ML model is trained on the plurality of sample 2D MR image slices of the sample volume of the sample MR scan as a single volume.

26. (canceled)

27. A computer implemented method for segmentation of FGT in a MR image, comprising:
feeding an MR image depicting a whole breast of a target subject that excludes an implant and on which no surgical removal has been performed into a FGT segmentation ML model; and
obtaining a segmentation of FGT of the MR image as an outcome of the FGT segmentation ML model,
wherein the FGT segmentation ML model is trained on a FGT segmentation training dataset of a plurality of FGT segmentation records, each FGT segmentation record including an MR image of a segmented whole breast of a control subject that excludes an implant and on which no surgical removal has been performed, and a ground truth comprising a segmentation of the FGT depicted in the segmented whole breast,
wherein the segmentation of the FGT is performed by:
co-registration between a first type of MR image of the whole breast of the control subject, and a second type of MR image of the whole breast of the control subject, the first type of MR image does not suppresses signals from fat tissue and the second type of MR image does suppress signals from fat tissue;
applying a first threshold to the first MR image such that voxels with gray scale values below the first threshold to generate a first mask;
applying a second threshold to the second MR image such that voxels with gray scale values above the second threshold to generate a second mask; and
labelling voxels that appear in the first mask and in the second mask as FGT.

28. A computer implemented method for training a FGT segmentation ML model for segmentation of FGT in a MR image, comprising:
creating a FGT segmentation training dataset of a plurality of FGT segmentation records, each FGT segmentation record including an MR image of a segmented whole breast of a control subject that excludes an implant and on which no surgical removal has been performed, and a ground truth comprising a segmentation of the FGT depicted in the segmented whole breast,
wherein the segmentation of the FGT is performed by:
co-registration between a first type of MR image of the whole breast of the control subject, and a second type of MR image of the whole breast of the control subject, the first type of MR image does not suppresses signals from fat tissue and the second type of MR image does suppress signals from fat tissue,
applying a first threshold to the first MR image such that voxels with gray scale values below the first threshold to generate a first mask,
applying a second threshold to the second MR image such that voxels with gray scale values above the second threshold to generate a second mask, and
labelling voxels that appear in the first mask and in the second mask as FGT; and
training the FGT segmentation ML model on the FGT segmentation training dataset.

29-30. (canceled)

31. The computer implemented method of claim 28, wherein the first type of MR image is a T2-weighted non-fat suppressed MR image or a T1-weighted non-fat suppressed MR image, and the second type of MR image is a T1-weighted fat suppressed MR image.

32. The computer implemented method of claim 28, further comprising:
creating a second FGT segmentation training dataset of a plurality of second FGT segmentation records, each second FGT segmentation record including:
a sample MR image of a sample subject depicting a sample residual breast with implant after surgical removal of breast tissue, and
a ground truth comprising a segmented sample FGT,
wherein the segmented sample FGT is segmented from the sample MR image by the segmenting the sample residual breast from the sample MR image, and segmenting the sample FGT from the segmented sample residual breast; and
training a second FGT segmentation ML model on the second FGT segmentation training dataset, wherein the second FGT segmentation ML model generates a target segmented FGT in response to an input of a target MR image depicting a residual breast with implant after surgical removal of breast tissue.

33. The computer implemented method of claim 28, wherein the FGT segmentation training dataset further includes a second set of FGT segmentation records, each record including an MR image of a residual breast on which surgery has been performed, with or without implant, and a ground truth comprising the segmentation of the FGT in the residual breast.
